(19) United States
(12) Patent Application Publication
(10) Pub. No.: US 2025/0380732 A1
(43) Pub. Date: Dec. 18, 2025

(54) AEROSOLISABLE FORMULATION

(71) Applicant: NICOVENTURES TRADING LIMITED, London (GB)

(72) Inventors: Theresa O'DONNELL, London (GB); Danielle TOWER, London (GB); Andrew James BURTON, London (GB); Nicole EAST, London (GB); Kelly O'ROURKE, London (GB); Samuel KAISER, Rio de Janeiro (BR); Marcelo Caetano Alexandre MARCELO, Rio de Janeiro (BR); Laura Fernanda Osmari VENDRAME, London (GB); Fabio Carrer ANDREIS, London (GB); Dominic Conrad WOODCOCK, London (GB); Maria Linhares de Andrade Rocha e SILVA, London (GB)

(21) Appl. No.: 18/877,779

(22) PCT Filed: Jun. 22, 2023
(86) PCT No.: PCT/GB2023/051637

§ 371 Date: Dec. 20, 2024

(30) Foreign Application Priority Data
Jun. 24, 2022 (GB) 2209298.5
Mar. 13, 2023 (GB) 2303663.5

(51) Int. Cl. A24B 15/24; A24B 15/167; A24B 15/30; A24B 15/40; A24F 40/10

(52) U.S. Cl. CPC A24B 15/243; A24B 15/167; A24B 15/301; A24B 15/303; A24B 15/403; A24F 40/10

(57) ABSTRACT

There is provided an aerosolisable formulation comprising (a) at least one active agent; (b) aerosol former material; (c) a modulator selected from a TRPA1 agonist, a TRPMS agonist, a TRPV1 agonist, a TRPV3 agonist, a TRPA1 antagonist, a TRPA1 inhibitor and combinations thereof, and (d) one or more binders.

### FIELD OF THE INVENTION

The present disclosure relates to an aerosolisable formulation, a method of forming the same, a device for forming the same and processes and uses of the same.

### BACKGROUND TO THE INVENTION

Electronic aerosol provision systems such as e-cigarettes generally contain a reservoir of liquid which is to be vaporised, typically containing a flavour or an active agent such as nicotine. When a user inhales on the device, a heater is activated to vaporise a small amount of liquid, which is therefore inhaled by the user.

The use of e-cigarettes in the UK has grown rapidly, and it has been estimated that there are now over a million people using them in the UK.

One challenge faced in providing such systems is to provide from the aerosol provision device an aerosol to be inhaled which provides consumers with an acceptable experience. Some consumers may prefer an e-cigarette that generates an aerosol that closely ‘mimics’ smoke inhaled from a tobacco product such as a cigarette. Aerosols from e-cigarettes and smoke from tobacco products such as cigarettes provides to the user a complex chain of flavour in the mouth, and if nicotine is present, nicotine absorption in the mouth and throat, followed by nicotine absorption in the lungs. These various aspects are described by users in terms of flavour, intensity/quality, impact, irritation/smoothness and reward. Flavour contributes to a number of these factors, and is strongly associated with flavour in the mouth and the provision of desirable taste and smell, whether mimicking the taste and smell of a tobacco product or providing alternative flavours. Reliably providing a particular taste and smell is made more difficult by the volatile and thermally sensitive nature of some flavours. Heating of flavour components in e-cigarettes may result in some flavours being degraded. This has a number of disadvantages. Flavours present in the liquid may be lost resulting in a diminished flavour experience for the user or the need to include in the liquid excess flavour at additional cost. Furthermore, if e-liquid contains multiple flavours and only some of these multiple flavours are degraded, this can adversely affect the balance of the taste and smell. Furthermore, degraded flavours may have an undesirable or “off taste”. Each of these factors, and their balance, can strongly contribute to consumer acceptability of an e-cigarette. Providing means to optimise the overall vaping experience is therefore desirable to e-cigarette manufacturers.

A further challenge facing such systems is the continued demand for harm reduction. Harm from cigarette and e-cigarette devices primarily comes from toxicants. Therefore, there is a desire to reduce the potential for the formation of toxicants.

### SUMMARY OF THE INVENTION

In one aspect there is provided an aerosolisable formulation or aerosol formulation comprising
(a) at least one active agent;
(b) a carrier selected from water, propylene glycol, glycerol and mixtures thereof; and
(c) a modulator selected from a TRPA1 agonist, a TRPM8 agonist, a TRPV1 agonist, a TRPV3 agonist, a TRPA1 antagonist, a TRPA1 inhibitor and combinations thereof.

In one aspect there is provided a process for forming an aerosol comprising
(a) at least one active agent;
(b) a carrier selected from water, propylene glycol, glycerol and mixtures thereof; and
(c) a modulator selected from a TRPA1 agonist, a TRPM8 agonist, a TRPV1 agonist, a TRPV3 agonist, a TRPA1 antagonist, a TRPA1 inhibitor and combinations thereof, the process comprising one of
   (A) aerosolising an aerosolisable formulation comprising
(a) at least one active agent;
(b) a carrier selected from water, propylene glycol, glycerol and mixtures thereof; and
(c) a modulator selected from a TRPA1 agonist, a TRPM8 agonist, a TRPV1 agonist, a TRPV3 agonist, a TRPA1 antagonist, a TRPA1 inhibitor and combinations thereof or
   (B) aerosolising an aerosolisable formulation comprising
(a) at least one active agent; and
(b) a carrier selected from water, propylene glycol, glycerol and mixtures thereof; aerosolising an aerosolisable formulation comprising
(c) a modulator selected from a TRPA1 agonist, a TRPM8 agonist, a TRPV1 agonist, a TRPV3 agonist, a TRPA1 antagonist, a TRPA1 inhibitor and combinations thereof; or
   (C) aerosolising an aerosolisable formulation comprising
(a) at least one active agent;
(b) aerosol former material;
(c) a modulator selected from a TRPA1 agonist, a TRPM8 agonist, a TRPV1 agonist, a TRPV3 agonist, a TRPA1 antagonist, a TRPA1 inhibitor and combinations thereof;
and
(d) one or more binders;
   or
   (D) aerosolising an aerosol generating composition comprising
(a) botanical material;
(b) an aerosol former; and
(c) a modulator selected from selected from a TRPA1 agonist, a TRPM8 agonist, a TRPV1 agonist, a TRPV3 agonist, a TRPA1 antagonist, a TRPA1 inhibitor and combinations thereof.

In one aspect there is provided a contained aerosolisable formulation comprising
(i) a container; and
(ii) an aerosolisable formulation, comprising
   (a) at least one active agent;
   (b) a carrier selected from water, propylene glycol, glycerol and mixtures thereof; and
   (c) a modulator selected from a TRPA1 agonist, a TRPM8 agonist, a TRPV1 agonist, a TRPV3 agonist, a TRPA1 antagonist, a TRPA1 inhibitor and combinations thereof.

In one aspect there is provided a kit comprising
(i) a first container; and
(ii) an aerosolisable formulation, comprising
   (a) at least one active agent; and
   (b) a carrier selected from water, propylene glycol, glycerol and mixtures thereof;
(iii) a second container; and
(iv) a modulator formulation comprising a modulator selected from a TRPA1 agonist, a TRPM8 agonist, a TRPV1 agonist, a TRPV3 agonist, a TRPA1 antagonist, a TRPA1 inhibitor and combinations thereof.

In one aspect there is provided a kit comprising
(1) a first component comprising
(i) a first container; and
(ii) an aerosolisable formulation, comprising
   (a) at least one active agent; and
   (b) aerosol former material;
(2) a second component comprising
   (i) a second container; and
   (ii) a modulator formulation comprising a modulator selected from a TRPA1 agonist, a TRPM8 agonist, a TRPV1 agonist, a TRPV3 agonist, a TRPA1 antagonist, a TRPA1 inhibitor and combinations thereof.

In one aspect there is provided an electronic aerosol provision system comprising:
(i) an aerosoliser or aerosol-generator for aerosolising formulation for inhalation by a user of the electronic aerosol provision system;
(ii) a power supply comprising a cell or battery for supplying power to the aerosoliser or aerosol-generator
(iii) an aerosolisable formulation, comprising at least
   (a) at least one active agent; and
   (b) a carrier selected from water, propylene glycol, glycerol and mixtures thereof;
(iv) a modulator selected from a TRPA1 agonist, a TRPM8 agonist, a TRPV1 agonist, a TRPV3 agonist, a TRPA1 antagonist, a TRPA1 inhibitor and combinations thereof.

In one aspect there is provided an electronic aerosol provision system comprising:
(i) an aerosoliser or aerosol-generator for aerosolising formulation for inhalation by a user of the electronic aerosol provision system;
(ii) a power supply comprising a cell or battery for supplying power to the aerosoliser or aerosol-generator
(iii) an aerosolisable formulation, comprising at least
   (a) at least one active agent; and
   (b) aerosol former material;
(iv) a modulator selected from a TRPA1 agonist, a TRPM8 agonist, a TRPV1 agonist, a TRPV3 agonist, a TRPA1 antagonist, a TRPA1 inhibitor and combinations thereof.

In one aspect there is provided use of a modulator for reducing the harshness when inhaled of an aerosolised formulation, wherein the modulator is selected from a TRPA1 agonist, a TRPM8 agonist, a TRPV1 agonist, a TRPV3 agonist, a TRPA1 antagonist, a TRPA1 inhibitor and combinations thereof; and
wherein the aerosolised formulation comprises
(a) at least one active agent; and
(b) a carrier selected from water, propylene glycol, glycerol and mixtures thereof.

In one aspect there is provided use of a modulator for reducing the harshness when inhaled of an aerosolised formulation,
wherein the modulator is selected from a TRPA1 agonist, a TRPM8 agonist, a TRPV1 agonist, a TRPV3 agonist, a TRPA1 agonist, a TRPA1 inhibitor and combinations thereof; and
wherein the aerosolised formulation comprises
(a) at least one active agent; and
(b) aerosol former material.

In one aspect there is provided an aerosolisable formulation or aerosol formulation comprising
(a) at least one active agent;
(b) a carrier selected from water, propylene glycol, glycerol and mixtures thereof; and
(c) a modulator selected from a TRPA1 agonist, a TRPM8 agonist, a TRPV1 agonist, a TRPV3 agonist, a TRPA1 antagonist, a TRPA1 inhibitor and combinations thereof; OR an aerosolisable formulation or aerosol formulation comprising
(a) at least one active agent;
(b) aerosol former material; and
(c) a modulator selected from a TRPA1 agonist, a TRPM8 agonist, a TRPV1 agonist, a TRPV3 agonist, a TRPA1 antagonist, a TRPA1 inhibitor and combinations thereof.

In one aspect there is provided an aerosolisable formulation comprising
(a) at least one active agent;
(b) aerosol former material;
(c) a modulator selected from a TRPA1 agonist, a TRPM8 agonist, a TRPV1 agonist, a TRPV3 agonist, a TRPA1 antagonist, a TRPA1 inhibitor and combinations thereof; and
(d) one or more binders.

In one aspect there is provided an aerosol generating composition comprising
(a) botanical material;
(b) an aerosol former; and
(c) a modulator selected from selected from a TRPA1 agonist, a TRPM8 agonist, a TRPV1 agonist, a TRPV3 agonist, a TRPA1 antagonist, a TRPA1 inhibitor and combinations thereof.

In one aspect there is provided a consumable for use in a non-combustible aerosol provision device, the consumable comprising an aerosolisable formulation composition as defined herein.

In one aspect there is provided a non-combustible aerosol provision system comprising the consumable of the present invention and a non-combustible aerosol provision device, the non-combustible aerosol provision device comprising an aerosol-generation device arranged to generate aerosol from the consumable when the consumable is used with the non-combustible aerosol provision device.

In one aspect there is provided use of an aerosolisable formulation composition as defined herein in a consumable for use with a non-combustible aerosol provision device, the non-combustible aerosol provision device comprising an aerosol-generation device arranged to generate aerosol from the consumable when the consumable is used with the non-combustible aerosol provision device.

### BRIEF DESCRIPTION OF THE DRAWINGS

The present invention will now be described in further detail by way of example only with reference to the accompanying figures in which:

FIG. 1 is a plot of the assessment of irritation in a PDT;

FIG. 2 is a plot of the assessment of impact in a PDT;

FIG. 3 is a plot of the assessment of flavour intensity in a PDT; and

FIG. 4 is a plot of the assessment of likeability in a PDT.

### DETAILED DESCRIPTION

As discussed herein in one aspect there is provided an aerosolisable formulation comprising
(a) at least one active agent;
(b) a carrier selected from water, propylene glycol, glycerol and mixtures thereof; and
(c) a modulator selected from a TRPA1 agonist, a TRPM8 agonist, a TRPV1 agonist, a TRPV3 agonist, a TRPA1 antagonist, a TRPA1 inhibitor and combinations thereof.

As will be understood by one skilled in the art, an “aerosolisable formulation” is a formulation that is capable of generating aerosol, for example when heated, irradiated or energized in any other way. An “aerosolisable formulation”, namely an aerosol-generating formulation, may, for example, be in the form of a solid, liquid or semi-solid (such as a gel) which may or may not contain an active substance and/or flavourants.

We have found that an advantageous system may be provided in which an aerosolised formulation contains modulators which act on particular receptors in the body. In particular, we have found that the provision in an aerosol of a modulator selected from a TRPA1 agonist, a TRPM8 agonist, a TRPV1 agonist, a TRPV3 agonist, a TRPA1 antagonist, a TRPA1 inhibitor and combinations thereof, may improve the sensorial properties of the aerosol when inhaled. More specifically, the presence of this modulator or modulators may reduce the harshness of the aerosol when inhaled.

For ease of reference, these and further aspects of the present invention are now discussed under appropriate section headings. However, the teachings under each section are not necessarily limited to each particular section.

### Modulator

As discussed herein, the present invention utilises a modulator selected from a TRPA1 agonist, a TRPM8 agonist, a TRPV1 agonist, a TRPV3 agonist, a TRPA1 antagonist, a TRPA1 inhibitor and combinations thereof. In one aspect, the modulator comprises at least one or more of a TRPA1 agonist, a TRPM8 agonist, a TRPV1 agonist, and a TRPV3 agonist. In one aspect, the modulator comprises at least a TRPA1 antagonist or a TRPA1 inhibitor.

In one aspect, the modulator comprises at least
(i) one or more of a TRPM8 agonist, a TRPV1 agonist, and a TRPV3 agonist; and
(ii) an TRPA1 antagonist or a TRPA1 inhibitor.

In one aspect, the modulator comprises at least (i) a TRPM8 agonist or a TRPV1 agonist; and (ii) a TRPA1 antagonist or a TRPA1 inhibitor. In one aspect, the modulator comprises at least (i) a TRPM1 agonist or a TRPV3 agonist; and (ii) a TRPA1 antagonist or a TRPA1 inhibitor. In one aspect, the modulator comprises at least (i) a TRPM3 agonist or a TRPV8 agonist; and (ii) a TRPA1 antagonist or a TRPA1 inhibitor.

It will be appreciated by one skilled in the art that each of the modulators is capable of binding to suitable receptors to act as the required agonist, antagonist or inhibitor. When a potential modulator interacts with a receptor then the interaction will have a given binding energy. The binding energy may be determined based on the follow equation:

$$F E {B}_{dock} \approx Δ {G}_{bind} = Δ {G}_{complex} - ( Δ {G}_{protein} + Δ {G}_{ligand} )$$

More specifically, the modulator and receptor involves several molecular interactions (e.g., van der Waals, electrostatic, hydrogen bonds, hydrophobic, and others) and physical-chemical complementarity among the receptor and the modulator. The protein structure file (receptor) may be retrieved from the Protein Data Bank and was described using electron microscopy model. The details are as follows:
TRPV1: PDB: 7LR0, https://www.rcsb.org/structure/7LR0, DOI: 10.2210/pdb7Ir0/pdb;
TRPV3: PDB: 6DVZ, https://www.rcsb.org/structure/6DVZ, DOI: 10.2210/pdb6dvz/pdb
TRPM8: PDB: 6NR2, https://www.rcsb.org/structure/6NR2, DOI: 10.2210/pdb6nr2/pdb
TRPA1 inhibitor: 6WJ5, https://www.rcsb.org/structure/6WJ5, DOI: 10.2210/pdb6wj5/pdb
TRPA1 antagonist: 7JUP, https://www.rcsb.org/structure/7JUP, DOI: 10.2210/pdb7jup/pdb
TRPA1 agonist: 6X2J, https://www.rcsb.org/structure/6X2J, DOI: 10.2210/pdb6X2J/pdb. The receptors hydrogen atoms may be added, according to appropriate hybridization geometry, to those atoms based on built-in modules to add partial charges, protonation states followed by bond orders assignment and set up rotatable bonds of the receptors. Active site pockets of a protein molecule are the sites where the potential modulators bind to a protein. To perform these experiments, one may use one simulation box that determinates the searching area for the modulators. This theoretical procedure may be performed using the TRPA1 agonist grid-box size with dimensions of X=20 Å, Y=20 Å, Z=20 Å and a center coordinates of X=179.3 Å, Y=146.94 Å, Z=191.79 Å to evaluate the modulator interactions. TRPA1 inhibitor analysis may be performed using the grid-box size with dimensions of X=20 Å, Y=20 Å, Z=20 Å and a center coordinates of X=156.75 Å, Y=139.94 Å, Z=185.30 Å. TRPA1 antagonist may be performed using the grid-box size with dimensions of X=20 Å, Y=20 Å, Z=20 Å and a center coordinates of X=62.58 Å, Y=63.50 Å, Z=86.13 Å. For TRPV3, this may be performed using the grid-box size with dimensions of X=20 Å, Y=20 Å, Z=20 Å and a center coordinates of X=88.45 Å, Y=107.91 Å, Z=122.14 Å. For TPV1, this may be performed using the grid-box size with dimensions of x=20 Å, y=20 Å, z=20 Å and a center coordinates of X=106.82 Å, Y=82.40 Å, Z=90.48 Å. For TRPM8, this may be performed using the grid-box size with dimensions of X=20 Å, Y=20 Å, Z=20 Å and a center coordinates of X=136.90 Å, Y=106.92 Å, Z=169.89 Å.

Gibbs free energy of binding (FEB in Kcal/mol) for complexes structures of a receptor-modulator may be calculated using a scoring function which approximates the chemical potentials (ΔG_{bind}).

The docking free energy of binding (FEB_{dock}) is defined by ΔG_{bind} values for all docked poses of the formed complexes (protein-modulators) and include the internal steric energy of a given protein and modulators which can be expressed as the sum of individual molecular mechanics terms of standard-chemical potentials.

$$F E {B}_{dock} \approx Δ {G}_{bind} = Δ {G}_{complex} - ( Δ {G}_{protein} + Δ {G}_{ligand} )$$

A more negative value of FEB (kcal/mol) implies a higher affinity between the receptor and the different types of modulator substrates. The docking simulation results may be categorized like energetically unfavorable when the Gibbs free energy of the formed complexes is ΔG_{bind} ≥0 kcal/mol, pointing either to extremely low or complete absence of binding affinity, otherwise they are categorized like having medium to high docking affinity,

The docking simulation results may be categorized like energetically unfavorable when the Gibbs free energy of the formed complexes is ΔG_{bind} ≥0 kcal/mol, pointing either to extremely low or complete absence of binding affinity. In summary, the FEB threshold is 0 kcal/mol. FEB values higher than 0 means an absence of affinity for the receptor. If FEB is lower than 0 kcal/mol, we starting have affinity for receptors. Lowest FEB values means a better affinity for receptor.

TRPA1 Agonist Docking Simulation

| Name | CAS RN | FEB_{dock} (kcal/mol) |
|---|---|---|
| Δ⁹-THCA | 23978-85-0 | −8.363 |
| CBC | 20675-51-8 | −8.216 |
| Δ⁸-THC | 5957-75-5 | −7.989 |
| CBNA | N/A | −7.941 |
| CBDA | 1244-58-2 | −7.757 |
| Reference (GNE551) * | N/A | −7.728 |
| Δ⁹-THC | 1972-08-3 | −7.594 |
| CBD | 13956-29-1 | −7.537 |
| CBN | 521-35-7 | −7.502 |
| Δ⁹-THCV | 31262-37-0 | −7.500 |
| CBDV | 24274-48-4 | −7.031 |
| CBCA | N/A | −6.997 |
| CBGA | 25555-57-1 | −6.934 |
| CBG | 25654-31-3 | −6.344 |
| Nicotine | 25162-00-9 | −5.444 |

* From crystal structure, 5-amino-1-[(4-bromo-2-fluorophenyl)methyl]-N-(2,5-dimethoxyphenyl)-1H-1,2,3-triazole-4-carboxamide

TRPV1 Docking Simulation

| Name | CAS RN | FEB_{dock} (kcal/mol) |
|---|---|---|
| Δ⁹-THCA | 23978-85-0 | −9.568 |
| CBDA | 1244-58-2 | −9.302 |
| Δ⁹-THC | 1972-08-3 | −9.119 |
| Δ⁹-THCV | 31262-37-0 | −9.051 |
| CBCA | N/A | −8.818 |
| CBGA | 25555-57-1 | −8.689 |
| CBNA | N/A | −8.680 |
| CBN | 521-35-7 | −8.650 |
| CBC | 20675-51-8 | −8.609 |
| Δ⁸-THC | 5957-75-5 | −8.602 |
| Reference (capsaicin)* | 404-86-4 | −8.089 |
| CBG | 25654-31-3 | −7.923 |
| CBDV | 24274-48-4 | −7.883 |
| CBD | 13956-29-1 | −7.842 |
| Nicotine | 25162-00-9 | −6.822 |

*From crystal structure, 8-methyl-N-vanillyl-6-nonenamide

Ab initio modelling indicates that the abovementioned compounds bind to agonist sites of both TRPA1 and TRPV1 (FEB_{dock} variation within +2.0 kcal/mol compared to reference ligand).

Sampled cannabinoids display improved affinity to both TRPA1 and TRPV1 relative to nicotine, and favorable interaction with key amino-acids residues, indicating that the binding site may be the same as for nicotine.

In one aspect, the TRPA1 antagonist or a TRPA1 inhibitor has a stronger binding affinity to the TRPA1 receptor than the binding affinity of the one or more of a TRPM8 agonist, a TRPV1 agonist, and a TRPV3 agonist to their respective receptor. In one aspect, the TRPA1 antagonist or a TRPA1 inhibitor has a stronger binding affinity to the TRPA1 receptor than the binding affinity of the TRPM8 agonist to the TRPM8 receptor. In one aspect, the TRPA1 antagonist or a TRPA1 inhibitor has a stronger binding affinity to the TRPA1 receptor than the binding affinity of the TRPV1 agonist to the TRPV1 receptor. In one aspect, the TRPA1 antagonist or a TRPA1 inhibitor has a stronger binding affinity to the TRPA1 receptor than the binding affinity of the TRPV3 agonist to the TRPV3 receptor. In one aspect, the TRPA1 antagonist or a TRPA1 inhibitor has a stronger binding affinity to the TRPA1 receptor than the binding affinity of the TRPM8 agonist to the TRPM8 receptor, the TRPV1 agonist to the TRPV1 receptor and the TRPV3 agonist to the TRPV3 receptor

In one aspect, the modulator comprises at least (i) a TRPM8 agonist and a TRPV1 agonist; and (ii) a TRPA1 antagonist or a TRPA1 inhibitor. In one aspect, the modulator comprises at least (i) a TRPM8 agonist and a TRPV3 agonist; and (ii) a TRPA1 antagonist or a TRPA1 inhibitor. In one aspect, the modulator comprises at least (i) a TRPM1 agonist and a TRPV3 agonist; and (ii) a TRPA1 antagonist or a TRPA1 inhibitor. In one aspect, the modulator comprises (i) a TRPM1 agonist, a TRPV3 agonist and a TRPV8 agonist; and (ii) a TRPA1 antagonist or a TRPA1 inhibitor.

It will be appreciated by one skilled in the art that a given compound may be a modulator of one or more of TRPM1, TRPV3, TRPV8 and TRPA1. In one aspect, the modulator utilised in the present invention is a single compound. In one aspect, the modulator of each of the one or more of TRPM1, TRPV3, TRPV8 and TRPA1 is a separate compound.

To modulate the activity of each of TRPM1, TRPV3, TRPV8 and TRPA1, the modulators may bind with any suitable binding site necessary to modulate the activity.

In one aspect, the TRPA1 antagonist binds with one or more sites selected from Arg852, Gln979, His983, Ile858, Leu982, Met978, Trp711, Val861, Val967, Ala836, Gln940, Ile837, Leu847, Leu848, Leu863, Leu867, Leu871, Met844, Phe841, Phe884, Phe947, Ser887,

Tyr840; and combinations thereof. In one aspect, the TRPA1 antagonist binds with one or more sites selected from Arg852, Gln979, His983, Ile858, Leu982, Met978, Trp711, Val861, Val967; and combinations thereof. In one aspect, in one aspect, the TRPA1 agonist binds with one or more sites selected from Ala836, Gln940, Ile837, Leu847, Leu848, Leu863, Leu867, Leu871, Met844, Phe841, Phe884, Phe947, Ser887, Tyr840; and combinations thereof.

In one aspect, the TRPM8 agonist binds with one or more sites selected from Arg1007, Arg841, Asn741, Asp781, Ile845, Leu1000, Phe738, Tyr1004, Tyr745, Val848; and combinations thereof.

In one aspect, the TRPV1 agonist binds with one or more sites selected from Ala548, Ala568, Ala667, Asn553, Glu572, Ile571, Ile663, Leu517, Leu555, Leu664, Leu671, Met549, Phe518, Phe545, Phe593, Ser514, Thr552, Tyr513, Tyr556; and combinations thereof.

In one aspect, the TRPV3 agonist binds with one or more sites selected from Arg693, His430, His426, His417, Leu420, Leu694, Leu429, Trp433, Thr421; and combinations thereof.

In one aspect, the TRPA1 inhibitor binds with one or more sites selected from Leu867, Leu870, Leu871, Ile946, Ser873 Thr873, Thr874, Phe944, Val948, Phe877, Ile878, Leu880, Leu881, Met912, Phe909, Thr908, Ile906, Ile905, Ile950, Leu956, Val942, Met953, Leu952, Phe884; and combinations thereof.

The modulator may be selected from any suitable compounds which provide the necessary modulating activity. In one aspect the modulator is selected from 3-Phenylpropionic acid; Acetic acid; Benzaldehyde; Benzoic acid; Benzyl Alcohol; 1-Butanol; Butyric acid; 4-Isopropylbenzaldehyde; 2-Methoxy-4-vinylphenol; Octanoic acid; Octanal; Guaiacol; Lactic acid; 3,4-Dihydrocoumarin; Ethanol; Glycerol; 1-Octanol; Phenylacetaldehyde; Phenylacetic acid; Propylene glycol; Propionic acid; Pyruvic acid; Dimethyl sulfide; 4-Methyl-5-thiazoleethanol; Vanillin; Menthol; Camphor; Eucalyptol; Decanoic acid; Eugenol; hexylresorcinol; Lauric acid; Triacetin; 2-Phenylethanol; Hexanal; 1-Pentanol; Bornyl acetate; Ethyl methylphenylglycidate; Triethyl citrate; Linalool; Isobutanol; Isobutyraldehyde; Isobutyric acid; 4-(2,5,6,6-Tetramethylcyclohex-2-en-1-yl)but-3-en-2-one; alpha-Pinene; Cyclotene; 3-2-(methylamino)benzoate; (1R,9S)-4,11,11-trimethyl-8-Methylindole; Methyl methylidenebicyclo[7.2.0]undec-4-ene; p-Menthan-3-one; Piperitone; Thymol; 2-Ethylphenol; 2,6-Dimethoxyphenol; 6-Methylquinoline; Methyl isoeugenol; 2-Methoxy-4-methylphenol; Methyl benzoate; Ethyl benzoate; 2-Methylbutyraldehyde; gamma-Butyrolactone; 2-Methylvaleric acid; Ethyl isobutyrate; Ethyl lactate; Furfuryl mercaptan; Acetophenone; Carveol; Carvone; gamma-Terpinene; 4′-Methoxyacetophenone; Methyl phenylacetate; Diphenyl ether; Ethyl phenylacetate; Isoamyl phenylacetate; Phenethyl phenylacetate; 2-Propenoic acid, 3-phenyl-, phenylmethyl ester; Phenethyl acetate; Phenethyl isobutyrate; 4-Methoxybenzyl acetate; gamma-Octalactone; Benzyl formate; Gamma-nonalactone; Gamma-undecalactone; 4-Methylbenzaldehyde; 1-Methoxy-4-methylbenzene; 4-Methoxybenzyl alcohol; gamma-Heptalactone; Ethyl propionate; 3-Methylvaleric acid; Diethyl malonate; Ethyl butyrate; Acetal; Propyl butyrate; Isoamyl propionate; (−)-Citronellol; Citronellal; Isoamyl butyrate; Ethyl heptanoate; Ethyl octanoate; Ethyl dodecanoate; Methyl hexanoate; Hydroxycitronellal; 2-Pentanone; gamma-Valerolactone; 2,6-Dimethylpyridine; 2,6-Dimethylpyrazine; Ethyl isovalerate; 4-Methylpyridine; 3-Methylpyridine; 2-Methylpyrazine; Butyl isovalerate; Butyl butyrate; Valeric acid; Propyl acetate; Ethyl formate; Isobutyl acetate; Isopropyl myristate; Ethyl decanoate; Diethyl sebacate; 2-Heptanone; Pentanal; Heptanoic acid; 1-Hexanol; 1-Heptanol; Nonenoic acid, methyl ester; Nonanoic acid; 2-Undecanone; Octyl acetate; Decanal; 1-Dodecanol; Dodecanal; Linalyl acetate; 2-Methylbutanoic acid; Maltol; Veratraldehyde; Piperonal; Ethyl vanillin; Methyl 4-methoxybenzoate; 4′-Methylacetophenone; Methyl anthranilate; 2-Methyl-1-butanol; Benzyl acetate; Phenethyl isovalerate; p-Tolyl acetate; Citronellol; Ethyl acetate; Ethyl acetoacetate; Hexanoic acid; Hexyl acetate; 1,4-Dimethoxybenzene; Citronellyl acetate; (7R,11R)-3,7,11,15-tetramethylhexadec-2-en-1-ol; 1,3-Dimethoxybenzene; 6-Methyl-5-hepten-2-one; Camphor, (1S,4S)-(−)-; 1,4-Cineole; 2-Cyclopenten-1-one, 3-methyl-2-(2Z)-2-pentenyl-; 2,4,5-Trimethylphenol; Carvacrol; Isovaleric acid; Gluconic acid; 2,4,6-Trimethylphenol; 4-Ethylpyridine; 3-Ethylpyridine; Ethyl valerate; Ethyl levulinate; Isobutyl butyrate; Pentyl hexanoate; Pentyl butyrate; Terpinen-4-ol; Terpinolene; Isobutyl isovalerate; 3-Methylbutanal; alpha-Angelica lactone; Methyl 2-furoate; 5-Methylfurfural; 3-Ethylphenol; Methyl butyrate; 2-Butenoic acid, ethyl ester; 4-Propylphenol; Isoamyl isovalerate; gamma-Caprolactone; 2,3,5-Trimethylphenol; delta-Octalactone; delta-Decalactone; gamma-Decalactone; delta-Dodecalactone; 2-Nonanone; delta-Hexalactone; Methyl 2-methylbutyrate; 2-Acetylpyrrole; Hexyl octanoate; 4-Acetylpyridine; 2-Acetylpyridine; 2,3,5,6-Tetramethylpyrazine; 2-Acetyl-5-methylfuran; beta-Pinene; 1-Hexen-3-one; Fenchol; Hex-3-enyl acetate; 3-Methylbutyl octanoate; 3-Methylbutyl hexanoate; I-Menthol; d-Carvone; gamma-Dodecalactone; alpha-Terpineol; Hexyl butyrate; 2-Methoxy-4-propylphenol; 2-Methoxy-3-methylpyrazine; 2-Pentenoic acid, 2-methyl-; 2-Methyltetrahydrofuran-3-one; 3-(Methylthio) propionaldehyde; delta-Nonalactone; beta-Ocimene; Sabinene; 1-Octen-3-OL; Neomenthol; Furaneol; Dimethyl trisulfide; 2,3-Hexanedione; Hex-3-enoic acid; Benzeneacetaldehyde, alpha-ethylidene-; Ethyl maltol; 4-(4-Hydroxyphenyl)-2-butanone; 2,3-Dimethylpyrazine; Linalyl oxide; Hexyl hexanoate; alpha-Caryophyllene; Ethyl 2-methylbutyrate; Butyl butyryllactate; Hexyl 2-methylbutanoate; Butanoic acid, 1,1-dimethyl-2-phenylethyl ester; 3-Ethyl-2,5-dimethylpyrazine; Bicyclo[7.2.0]undec-4-ene, 4, 11,11-trimethyl-8-methylene-, (1S,4E,9R)—; 2-Ethyl-3,5-dimethylpyrazine; Menthone; 2,3,5-Trimethylpyrazine; 2-Ethyl-3-methylpyrazine; 2,3-Diethylpyrazine; Menthyl acetate; Propylidene phthalide; Acetylpyrazine; alpha-Amylcinnamaldehyde; 2-Propenoic acid, 3-phenyl-, 3-phenyl-2-propenyl ester; 3-Phenyl-1-propanol; 4-Ethylphenol; 4-Methoxybenzaldehyde; 4-Heptanone; Ethyl nonanoate; 2,5-Dimethylpyrazine; Isoamyl alcohol; Ethyl hexanoate; Allyl hexanoate; Butyl acetate; Isoamyl acetate; Ethyl tetradecanoate; Nonanal; Linalyl benzoate; beta-damascone; 5-Methyl-6,7-dihydro-5H-cyclopenta[b]pyrazine; 2-Isobutyl-3-methoxypyrazine; 2-Ethyl-4-hydroxy-5-methyl-3 (2H)-furanone; 2,6-Nonadien-1-ol; Hex-3-enyl formate; 5,6,7,8-Tetrahydroquinoxaline; cis-3-Hexenyl 3-methylbutanoate; Massoia lactone; (+)-Isomenthol; Isobutyl phenylacetate; Benzyl phenylacetate; 4-(4-Methoxyphenyl)-2-butanone; 2,6-Dimethyl-5-heptenal; 1-Penten-3-one, 1-[(1R)-2,6,6-trimethyl-2-cyclohexen-1-yl]-, (1E)-; Furfuryl propionate; Hexyl formate; 5-Ethyl-3-hydroxy-4-methylfuran-2 (5H)-one; 2-Hydroxy-4-methylbenzaldehyde; 6-Hexyltetrahydro-2H-pyran-2-one; 3-Methylcyclopentane-1,2-dione; d-Piperitone; 3-Hexenyl 2-methylbutyrate; Ethyl 3-(methylthio) propionate; 3,4-Dimethyl-1,2-cyclopentanedione; 3,5-Dimethyl-1,2-cyclopentanedione; Methyl 3-(methylthio) propionate; 5-Methyl-2-thiophenecarboxaldehyde; 5-Methylquinoxaline; 2-Isopropyl-4-methylthiazole; Butyl 2-methylbutyrate; 2,4-Dimethylbenzaldehyde; Theaspirane; p-Mentha-8-thiol-3-one; p-Cresyl isovalerate; Orange oil; Linalyl butyrate; 1-Phenylethyl acetate; 2,6,6-Trimethyl-2-cyclohexene-1,4-dione; S-Methyl butanethioate; 4-Ethyl-2-methoxyphenol; (−)-beta-Bourbonene; Ethyl 3-hydroxybutyrate; 3-Ethyl-2-hydroxy-2-cyclopenten-1-one; 3-Hydroxy-4,5-dimethylfuran-2 (5H)-one; Whiskey lactone; N-Ethyl-p-menthane-3-carboxamide; Borneol; 3-(Methylthio)-1-hexanol; (+)-Linalool; 2-Methoxybenzyl alcohol; 3-Propylphenol; (+)-Isomenthone; 3-Methylbutyl pentanoate; 2-Methoxy-6-methylphenol; Ethyl (methylthio)acetate; Rhodinol; (+)-alpha-Pinene; Isoamyl lactate; Nicotine; 2-Mercaptopinane; 1,6-Cyclodecadiene, 1-methyl-5-methylene-8-(1-methylethyl)-; (+)-Camphene; Spathulenol; (−)-Bornyl acetate; 5-Ethyl-4-hydroxy-2-methylfuran-3 (2H)-one; 3,5,5-Trimethylcyclohexane-1,2-dione; 1-Oxacycloheptadec-10-en-2-one; (3R)-3,7-dimethyloct-6-en-1-ol; 6-Methyl-2-(oxiran-2-yl)hept-5-en-2-ol; Isobutyl 2-methylbutyrate; Methyl dihydrojasmonate; Ambrox; 2-Cyclohexen-1-one, 4-(2-butenylidene)-3,5,5-trimethyl-; L-Lactic acid; alpha-Terpinyl acetate; 3-Ethylcyclopentane-1,2-dione; 5-Nonen-2-one; Isobutyl decanoate; 2-Ethoxy-3-methylpyrazine; Tiglic acid; 3,7,11,15-Tetramethylhexadec-2-en-1-ol; 3-Methyl-2-butene-1-thiol; (+)-Nicotine; D-Camphor; (+)-Menthol; 2-Ethyl-6-methoxyphenol; L-Menthyl acetate; Guaiol; Oxacyclohexadecan-2-one; beta-Bourbonene; Ethyl crotonate; (+)-Neomenthol; (1R,2S,4R)-Borneol; Bicyclo[2.2.1]heptan-2-ol, 1,7,7-trimethyl-, exo-; (−)-Carvone; D-Limonene; (−)-Camphene; (−)-beta-Pinene; (−)-alpha-Pinene; (1S,2R,4S)-(−)-Bornyl acetate; (−) -Linalool; (+)-Menthone; (−)-alpha-Terpineol; (−)-Camphor; L (+)-Tartaric acid; Cinnamic acid; liquiritin; exo-1,7,7-Trimethylbicyclo (2.2.1) heptan-2-ol; Hexyl lactate; 2-sec-Butyl-3-methoxypyrazine; 2-Acetylthiazole; beta-Spathulenol; Isobutyl acetoacetate; 1-Methyl-2-oxopropyl butyrate; 3-Methylnonane-2,4-dione; 2,6,8-Decatrienamide, N-(2-methylpropyl)-, (2E,6Z,8E)-; 3-Mercaptohexyl hexanoate; 3,7-Dimethyloct-7-en-1-ol; 2-Methyl-1-[1-(2-methylbutoxy)ethoxy]butane; Menthyl isovalerate; 4-Methyl-2-phenyl-1,3-dioxolane; Cinnamaldehyde; Methyl cinnamate; Isoborneol, acetate; Anethole; Geraniol; Ethyl cinnamate; Methylisoeugenol; Citral; beta-lonone; Sorbic acid; trans-2,cis-6-Nonadienal; Neral; Nerol; Sumatra camphor; Isoeugenol; (−)-Borneol; Nootkatone; Jasmone; (E)-jasmone; Neryl acetate; Geranyl acetate; Geranylacetone; Cinnamic acid, cinnamyl ester; Cinnamyl cinnamate; (Z)-1-(2,6,6-Trimethyl-1-cyclohexen-1-yl)-2-buten-1-one; Amylcinnamaldehyde; 2-Benzylideneheptanal; (−)-Neoisomenthol; Caryophyllene oxide; trans-2-Hexenyl acetate; 5-Ethyl-2-methoxyphenol; 3-Methylcyclohexane-1,2-dione; (7E)-1-oxacycloheptadec-7-en-2-one; 4-Hydroxy-5-methylfuran-3 (2H)-one; Vanillyl butyl ether; Isopentyl cinnamate; Benzyl cinnamate; Phytol; cis-3-Hexen-1-ol; 2-Hexenal; beta-Caryophyllene; Humulene; 3-Buten-2-one, 4-(2,5,6,6-tetramethyl-2-cyclohexen-1-yl)-; Isocaryophyllene; (E)-beta-ocimene; alpha-lonone; Geranyl formate; Cinnamyl acetate; trans-2-Hexenoic acid; 3-Hexenoic acid; trans-3-Hexen-1-ol; Nerolidol; Cinnamyl alcohol; 2-Hexen-1-OL; (1Z,4E,8E)-2,6,6,9-tetramethylcycloundeca-1,4,8-triene; (2E,6E)-Nona-2,6-dien-1-ol; (Z)-3,7,11,15-tetramethylhexadec-2-en-1-ol; (4Z)-4, 11, 11-trimethyl-8-methylidenebicyclo[7.2.0]undec-4-ene; cis-3-Hexenyl butyrate; cis-3-Hexenyl hexanoate; trans-3-Hexenyl acetate; Spilanthol; ethyl (2Z)-but-2-enoate; Vanitrope; trans-Caryophyllene; cis-3-Hexenoic acid; Oxacycloheptadec-10-en-2-one; Geranyl butyrate; 2-Nonenoic acid, methyl ester; 5-Methyl-2-hepten-4-one; cis-4-Decenal; Ethyl 3-hexenoate; cis-6-Nonenal; (Z)-Non-6-en-1-ol; cis-4-Heptenal; (2E,6Z)-Nona-2,6-dien-1-ol; cis-3-Hexenyl acetate; 2-Octenoic acid, ethyl ester; cis-3-Hexenyl 2-methylbutanoate; cis-3-Hexenyl formate; 2-Octen-4-one; 2-Methyl-2-pentenoic acid; Damascenone; 3,7,11, 15-Tetramethyl-2-hexadecen-1-OL; Methyl 2-nonenoate; 6-Methyl-3,5-heptadien-2-one; (2Z)-2-Phenyl-2-butenal; alpha-Irone; 3-Propylidenephthalide; 1-Methyl-5-methylene-8-(1-methylethyl)-1,6-cyclodecadiene; trans-4-Decen-1-al; 2-Chloro-2-(phenylhydrazono) acetic acid ethyl ester; 3-Hexenyl 2-methylbutanoate; Propylidenephthalide; 3-Hexen-1-ol, formate; (E)-2-epi-beta-caryophyllene; 2-Phenyl-2-butenal; Isomenthone; 2-Pentenoic acid, 2-methyl-, (2Z)-; Megastigmatrienone A; (1E,4E,8E)-2,6,6,9-tetramethylcycloundeca-1,4,8-triene; Oxacycloheptadec-7-en-2-one, (7Z)-; (+)-Borneol; (−)-Neomenthol; Exo-1,7,7-trimethylbicyclo[2.2.1]hept-2-yl acetate; (+)-Isoborneol; (+)-Camphor; (2E,6Z,8E,10E)-N-(2-Hydroxy-2-methylpropyl) dodeca-2,6,8,10-tetraenamide; Hydroxy-Beta-Sanshool; (+)-beta-Pinene; Ambroxan; Cubebol; (2e,6e,8e)-n-(2-Methylpropyl)-2,6,8-decatrienamide; Oxacycloheptadec-10-en-2-one, (E)-; Pichtosin; (1S)-6,6-Dimethyl-2-methylenebicyclo[3.1.1]heptane; (1R)-(−)-Menthyl acetate; (2S,5R)-5-Methyl-2-(propan-2-yl)cyclohexyl acetate; Spatulenol; Benzyl alcohol, cinnamate; (2E,6E,8Z)—N-(2-methylpropyl) deca-2,6,8-trienamide; (S)-alpha-Methylionone; Methyl-alpha-ionone; (1S)-1,7,7-trimethylbicyclo[2.2.1]heptan-2-ol; 4-(But-2-en-1-ylidene)-3,5,5-trimethylcyclohex-2-enone; Ethyl 3-(methylthio) butyrate; Bicyclo[3.1.1]heptane, 6,6-dimethyl-2-methylene-, (1S,5S)—; (2Z,6E)-2,6-dimethyl-10-methylidenedodeca-2,6,11-trienal; (2S,4R)-1,7,7-trimethylbicyclo[2.2.1]heptan-2-ol; Hydroxy-alpha-sanshool; N-(2-Hydroxy-2-methylpropyl) dodeca-2,6,8, 10-tetraenamide; Ascorbic acid; (S, 1Z,6Z)-8-Isopropyl-1-methyl-5-methylenecyclodeca-1,6-diene; Jasmine oil; ALPHA-CARYOPHYLLENE; 3-Phenylpropyl homovanillate; 1,4,8-Cycloundecatriene, 2,6,6,9-tetramethyl-, (1E,4E,8E)-; (1E,8E)-2,6,6,9-tetramethylcycloundeca-1,4,8-triene; (1R,2S,6S,7R,8S)-1-methyl-5-methylidene-8-propan-2-yltricyclo[5.3.0.02,6]decane; (8E,10E)-N-(2-hydroxy-2-methylpropyl) dodeca-2,6,8,10-tetraenamide; and mixtures thereof.

In one aspect, the TRPA1 antagonist is selected from cinnamic acid, cinnamyl ester; hexylresorcinol; I-menthol; 2-ethyl-6-methoxyphenol; linalyl oxide; 2-methoxy-6-methylphenol; methyl 2-(methylamino)benzoate; eucalyptol; and mixtures thereof.

In one aspect, the TRPM8 agonist is selected from (1Z,4E,8E)-2,6,6,9-tetramethylcycloundeca-1,4,8-triene; beta-caryophyllene; liquiritin; ambrox; (E)-2-epi-beta-caryophyllene; bicyclo[7.2.0]undec-4-ene, 4,11,11-trimethyl-8-methylene-, (1R,4E,9S)—; (4Z)-4, 11,11-trimethyl-8-methylidenebicyclo[7.2.0]undec-4-ene; bicyclo[7.2.0]undec-4-ene, 4,11,11-trimethyl-8-methylene-, (1S,4E,9R)—; isocaryophyllene; ambroxan; spatulenol; caryophyllene oxide; (−)-beta-bourbonene; oxacycloheptadec-10-en-2-one, (E)-; cubebol; trans-caryophyllene; gamma-dodecalactone; hydroxy-alpha-sanshool; citronellyl acetate; (2E,6Z,8E,10E)-N-(2-Hydroxy-2-methylpropyl) dodeca-2,6,8, 10-tetraenamide; (8E,10E)-N-(2-hydroxy-2-methylpropyl) dodeca-2,6,8, 10-tetraenamide; N-(2-Hydroxy-2-methylpropyl) dodeca-2,6,8, 10-tetraenamide; hydroxy-beta-sanshool; 2-hexenal; and mixtures thereof.

In one aspect, the TRPV1 agonist is selected from phenethyl phenylacetate; 2-propenoic acid, 3-phenyl-, 3-phenyl-2-propenyl ester; theaspirane; (7R, 11R)-3,7,11,15-tetramethylhexadec-2-en-1-ol; 4-(2,5,6,6-tetramethylcyclohex-2-en-1-yl)but-3-en-2-one; cinnamyl cinnamate; alpha-irone; beta-spathulenol; spathulenol; phytol; 1-penten-3-one, 1-[(1R)-2,6,6-trimethyl-2-cyclohexen-1-yl]-, (1E)-; (S)-alpha-Methylionone; Guaiol; 3,7, 11, 15-tetramethylhexadec-2-en-1-ol; 3,7,11,15-tetramethyl-2-hexadecen-1-ol; (Z)-3,7,11,15-tetramethylhexadec-2-en-1-ol; vanillyl butyl ether; 4-ethyl-2-methoxyphenol; 6-methyl-2-(oxiran-2-yl)hept-5-en-2-ol; 2-methoxy-4-methylphenol; 2-methoxy-4-vinylphenol; benzyl cinnamate; 2-propenoic acid; 3-phenyl-, phenylmethyl ester; and mixtures thereof.

In one aspect, the TRPV3 agonist is selected from ethyl vanillin; 2,6-dimethoxyphenol; vanillin; and mixtures thereof.

In one aspect, the TRPA1 inhibitor is selected from 3-phenylpropyl homovanillate; benzyl alcohol, cinnamate; benzyl phenylacetate; alpha-amylcinnamaldehyde; linalyl benzoate; phenethyl isovalerate; carvacrol; thymol; 5-ethyl-2-methoxyphenol; vanitrope; and mixtures thereof.

In one aspect, the TRPA1 antagonist is selected from cinnamyl cinnamate, liquiritin, 3-phenylpropyl homovanillate, benzyl cinnamate, caryophyllene (beta-), phenethyl phenylacetate, hydroxy-alpha-sanshool, phytol, bourbonene (beta) and mixtures thereof.

In one aspect, the TRPA1 inhibitor is selected from cinnamyl cinnamate, citronellyl acetate, eucalyptol, 2-hexenal, dodecalactone (gamma-), menthol (I-), spathulenol, bourbonene (beta), benzyl cinnamate, caryophyllene oxide, caryophyllene (beta-), alpha-irone, phenethyl phenylacetate, methyl-alpha-ionone, phytol and mixtures thereof.

In one aspect, the modulator is at least caryophyllene oxide, benzyl cinnamate, or a mixture thereof.

In one aspect, the modulator is at least a mixture of caryophyllene oxide and benzyl cinnamate.

In one aspect the total amount of modulator present is no greater than 20 wt % based on the aerosolisable formulation or based on the aerosol formulation or based on the aerosolised formulation. In one aspect the total amount of modulator present is from 0.01 to 20 wt % based on the aerosolisable formulation or based on the aerosol formulation or based on the aerosolised formulation. In one aspect the total amount of modulator present is from 0.02 to 20 wt % based on the aerosolisable formulation or based on the aerosol formulation or based on the aerosolised formulation. In one aspect the total amount of modulator present is from 0.05 to 20 wt % based on the aerosolisable formulation or based on the aerosol formulation or based on the aerosolised formulation. In one aspect the total amount of modulator present is from 0.08 to 20 wt % based on the aerosolisable formulation or based on the aerosol formulation or based on the aerosolised formulation. In one aspect the total amount of modulator present is from 0.1 to 20 wt % based on the aerosolisable formulation or based on the aerosol formulation or based on the aerosolised formulation. In one aspect the total amount of modulator present is no greater than 15 wt % based on the aerosolisable formulation or based on the aerosol formulation or based on the aerosolised formulation. In one aspect the total amount of modulator present is from 0.01 to 15 wt % based on the aerosolisable formulation or based on the aerosol formulation or based on the aerosolised formulation. In one aspect the total amount of modulator present is from 0.02 to 15 wt % based on the aerosolisable formulation or based on the aerosol formulation or based on the aerosolised formulation. In one aspect the total amount of modulator present is from 0.05 to 15 wt % based on the aerosolisable formulation or based on the aerosol formulation or based on the aerosolised formulation. In one aspect the total amount of modulator present is from 0.08 to 15 wt % based on the aerosolisable formulation or based on the aerosol formulation or based on the aerosolised formulation. In one aspect the total amount of modulator present is no greater than 10 wt % based on the aerosolisable formulation or based on the aerosol formulation or based on the aerosolised formulation. In one aspect the total amount of modulator present is from 0.01 to 10 wt % based on the aerosolisable formulation or based on the aerosol formulation or based on the aerosolised formulation. In one aspect the total amount of modulator present is from 0.02 to 10 wt % based on the aerosolisable formulation or based on the aerosol formulation or based on the aerosolised formulation. In one aspect the total amount of modulator present is from 0.05 to 10 wt % based on the aerosolisable formulation or based on the aerosol formulation or based on the aerosolised formulation. In one aspect the total amount of modulator present is from 0.08 to 10 wt % based on the aerosolisable formulation or based on the aerosol formulation or based on the aerosolised formulation. In one aspect the total amount of modulator present is no greater than 8 wt % based on the aerosolisable formulation or based on the aerosol formulation or based on the aerosolised formulation. In one aspect the total amount of modulator present is from 0.01 to 8 wt % based on the aerosolisable formulation or based on the aerosol formulation or based on the aerosolised formulation. In one aspect the total amount of modulator present is from 0.02 to 8 wt % based on the aerosolisable formulation or based on the aerosol formulation or based on the aerosolised formulation. In one aspect the total amount of modulator present is from 0.05 to 8 wt % based on the aerosolisable formulation or based on the aerosol formulation or based on the aerosolised formulation. In one aspect the total amount of modulator present is from 0.08 to 8 wt % based on the aerosolisable formulation or based on the aerosol formulation or based on the aerosolised formulation. In one aspect the total amount of modulator present is no greater than 6 wt % based on the aerosolisable formulation or based on the aerosol formulation or based on the aerosolised formulation. In one aspect the total amount of modulator present is from 0.01 to 6 wt % based on the aerosolisable formulation or based on the aerosol formulation or based on the aerosolised formulation. In one aspect the total amount of modulator present is from 0.02 to 6 wt % based on the aerosolisable formulation or based on the aerosol formulation or based on the aerosolised formulation. In one aspect the total amount of modulator present is from 0.05 to 6 wt % based on the aerosolisable formulation or based on the aerosol formulation or based on the aerosolised formulation. In one aspect the total amount of modulator present is from 0.08 to 6 wt % based on the aerosolisable formulation or based on the aerosol formulation or based on the aerosolised formulation. In one aspect the total amount of modulator present is from 0.01 to 5 wt % based on the aerosolisable formulation or based on the aerosol formulation or based on the aerosolised formulation. In one aspect the total amount of modulator present is from 0.02 to 5 wt % based on the aerosolisable formulation or based on the aerosol formulation or based on the aerosolised formulation. In one aspect the total amount of modulator present is from 0.05 to 5 wt % based on the aerosolisable formulation or based on the aerosol formulation or based on the aerosolised formulation. In one aspect the total amount of modulator present is from 0.08 to 5 wt % based on the aerosolisable formulation or based on the aerosol formulation or based on the aerosolised formulation. In one aspect the total amount of modulator present is no greater than 4 wt % based on the aerosolisable formulation or based on the aerosol formulation or based on the aerosolised formulation. In one aspect the total amount of modulator present is from 0.01 to 4 wt % based on the aerosolisable formulation or based on the aerosol formulation or based on the aerosolised formulation. In one aspect the total amount of modulator present is from 0.02 to 4 wt % based on the aerosolisable formulation or based on the aerosol formulation or based on the aerosolised formulation. In one aspect the total amount of modulator present is from 0.05 to 4 wt % based on the aerosolisable formulation or based on the aerosol formulation or based on the aerosolised formulation. In one aspect the total amount of modulator present is from 0.08 to 4 wt % based on the aerosolisable formulation or based on the aerosol formulation or based on the aerosolised formulation. In one aspect the total amount of modulator present is no greater than 3 wt % based on the aerosolisable formulation or based on the aerosol formulation or based on the aerosolised formulation. In one aspect the total amount of modulator present is from 0.01 to 3 wt % based on the aerosolisable formulation or based on the aerosol formulation or based on the aerosolised formulation. In one aspect the total amount of modulator present is from 0.02 to 3 wt % based on the aerosolisable formulation or based on the aerosol formulation or based on the aerosolised formulation. In one aspect the total amount of modulator present is from 0.05 to 3 wt % based on the aerosolisable formulation or based on the aerosol formulation or based on the aerosolised formulation. In one aspect the total amount of modulator present is from 0.08 to 3 wt % based on the aerosolisable formulation or based on the aerosol formulation or based on the aerosolised formulation. In one aspect the total amount of modulator present is no greater than 2 wt % based on the aerosolisable formulation or based on the aerosol formulation or based on the aerosolised formulation. In one aspect the total amount of modulator present is from 0.01 to 2 wt % based on the aerosolisable formulation or based on the aerosol formulation or based on the aerosolised formulation. In one aspect the total amount of modulator present is from 0.02 to 2 wt % based on the aerosolisable formulation or based on the aerosol formulation or based on the aerosolised formulation. In one aspect the total amount of modulator present is from 0.05 to 2 wt % based on the aerosolisable formulation or based on the aerosol formulation or based on the aerosolised formulation. In one aspect the total amount of modulator present is from 0.08 to 2 wt % based on the aerosolisable formulation or based on the aerosol formulation or based on the aerosolised formulation. In one aspect the total amount of modulator present is no greater than 1 wt % based on the aerosolisable formulation or based on the aerosol formulation or based on the aerosolised formulation. In one aspect the total amount of modulator present is from 0.01 to 1 wt % based on the aerosolisable formulation or based on the aerosol formulation or based on the aerosolised formulation. In one aspect the total amount of modulator present is from 0.02 to 1 wt % based on the aerosolisable formulation or based on the aerosol formulation or based on the aerosolised formulation. In one aspect the total amount of modulator present is from 0.05 to 1 wt % based on the aerosolisable formulation or based on the aerosol formulation or based on the aerosolised formulation. In one aspect the total amount of modulator present is from 0.08 to 1 wt % based on the aerosolisable formulation or based on the aerosol formulation or based on the aerosolised formulation. In one aspect the total amount of modulator present is from 0.1 to 1 wt % based on the aerosolisable formulation or based on the aerosol formulation or based on the aerosolised formulation.

In one aspect caryophyllene oxide and benzyl cinnamate are present in a combined amount of no greater than 20 wt % based on the aerosolisable formulation or based on the aerosol formulation or based on the aerosolised formulation. In one aspect caryophyllene oxide and benzyl cinnamate are present in a combined amount of from 0.01 to 20 wt % based on the aerosolisable formulation or based on the aerosol formulation or based on the aerosolised formulation. In one aspect caryophyllene oxide and benzyl cinnamate are present in a combined amount of from 0.02 to 20 wt % based on the aerosolisable formulation or based on the aerosol formulation or based on the aerosolised formulation. In one aspect caryophyllene oxide and benzyl cinnamate are present in a combined amount of from 0.05 to 20 wt % based on the aerosolisable formulation or based on the aerosol formulation or based on the aerosolised formulation. In one aspect caryophyllene oxide and benzyl cinnamate are present in a combined amount of from 0.08 to 20 wt % based on the aerosolisable formulation or based on the aerosol formulation or based on the aerosolised formulation. In one aspect caryophyllene oxide and benzyl cinnamate are present in a combined amount of from 0.1 to 20 wt % based on the aerosolisable formulation or based on the aerosol formulation or based on the aerosolised formulation. In one aspect caryophyllene oxide and benzyl cinnamate are present in a combined amount of no greater than 15 wt % based on the aerosolisable formulation or based on the aerosol formulation or based on the aerosolised formulation. In one aspect caryophyllene oxide and benzyl cinnamate are present in a combined amount of from 0.01 to 15 wt % based on the aerosolisable formulation or based on the aerosol formulation or based on the aerosolised formulation. In one aspect caryophyllene oxide and benzyl cinnamate are present in a combined amount of from 0.02 to 15 wt % based on the aerosolisable formulation or based on the aerosol formulation or based on the aerosolised formulation. In one aspect caryophyllene oxide and benzyl cinnamate are present in a combined amount of from 0.05 to 15 wt % based on the aerosolisable formulation or based on the aerosol formulation or based on the aerosolised formulation. In one aspect caryophyllene oxide and benzyl cinnamate are present in a combined amount of from 0.08 to 15 wt % based on the aerosolisable formulation or based on the aerosol formulation or based on the aerosolised formulation. In one aspect caryophyllene oxide and benzyl cinnamate are present in a combined amount of no greater than 10 wt % based on the aerosolisable formulation or based on the aerosol formulation or based on the aerosolised formulation. In one aspect caryophyllene oxide and benzyl cinnamate are present in a combined amount of from 0.01 to 10 wt % based on the aerosolisable formulation or based on the aerosol formulation or based on the aerosolised formulation. In one aspect caryophyllene oxide and benzyl cinnamate are present in a combined amount of from 0.02 to 10 wt % based on the aerosolisable formulation or based on the aerosol formulation or based on the aerosolised formulation. In one aspect caryophyllene oxide and benzyl cinnamate are present in a combined amount of from 0.05 to wt % based on the aerosolisable formulation or based on the aerosol formulation or based on the aerosolised formulation. In one aspect caryophyllene oxide and benzyl cinnamate are present in a combined amount of from 0.08 to 10 wt % based on the aerosolisable formulation or based on the aerosol formulation or based on the aerosolised formulation. In one aspect caryophyllene oxide and benzyl cinnamate are present in a combined amount of no greater than 8 wt % based on the aerosolisable formulation or based on the aerosol formulation or based on the aerosolised formulation. In one aspect caryophyllene oxide and benzyl cinnamate are present in a combined amount of from 0.01 to 8 wt % based on the aerosolisable formulation or based on the aerosol formulation or based on the aerosolised formulation. In one aspect caryophyllene oxide and benzyl cinnamate are present in a combined amount of from 0.02 to 8 wt % based on the aerosolisable formulation or based on the aerosol formulation or based on the aerosolised formulation. In one aspect caryophyllene oxide and benzyl cinnamate are present in a combined amount of from 0.05 to 8 wt % based on the aerosolisable formulation or based on the aerosol formulation or based on the aerosolised formulation. In one aspect caryophyllene oxide and benzyl cinnamate are present in a combined amount of from 0.08 to 8 wt % based on the aerosolisable formulation or based on the aerosol formulation or based on the aerosolised formulation. In one aspect caryophyllene oxide and benzyl cinnamate are present in a combined amount of no greater than 6 wt % based on the aerosolisable formulation or based on the aerosol formulation or based on the aerosolised formulation. In one aspect caryophyllene oxide and benzyl cinnamate are present in a combined amount of from 0.01 to 6 wt % based on the aerosolisable formulation or based on the aerosol formulation or based on the aerosolised formulation. In one aspect caryophyllene oxide and benzyl cinnamate are present in a combined amount of from 0.02 to 6 wt % based on the aerosolisable formulation or based on the aerosol formulation or based on the aerosolised formulation. In one aspect caryophyllene oxide and benzyl cinnamate are present in a combined amount of from 0.05 to 6 wt % based on the aerosolisable formulation or based on the aerosol formulation or based on the aerosolised formulation. In one aspect caryophyllene oxide and benzyl cinnamate are present in a combined amount of from 0.08 to 6 wt % based on the aerosolisable formulation or based on the aerosol formulation or based on the aerosolised formulation. In one aspect caryophyllene oxide and benzyl cinnamate are present in a combined amount of from 0.01 to 5 wt % based on the aerosolisable formulation or based on the aerosol formulation or based on the aerosolised formulation. In one aspect caryophyllene oxide and benzyl cinnamate are present in a combined amount of from 0.02 to 5 wt % based on the aerosolisable formulation or based on the aerosol formulation or based on the aerosolised formulation. In one aspect caryophyllene oxide and benzyl cinnamate are present in a combined amount of from 0.05 to 5 wt % based on the aerosolisable formulation or based on the aerosol formulation or based on the aerosolised formulation. In one aspect caryophyllene oxide and benzyl cinnamate are present in a combined amount of from 0.08 to 5 wt % based on the aerosolisable formulation or based on the aerosol formulation or based on the aerosolised formulation. In one aspect caryophyllene oxide and benzyl cinnamate are present in a combined amount of no greater than 4 wt % based on the aerosolisable formulation or based on the aerosol formulation or based on the aerosolised formulation. In one aspect caryophyllene oxide and benzyl cinnamate are present in a combined amount of from 0.01 to 4 wt % based on the aerosolisable formulation or based on the aerosol formulation or based on the aerosolised formulation. In one aspect caryophyllene oxide and benzyl cinnamate are present in a combined amount of from 0.02 to 4 wt % based on the aerosolisable formulation or based on the aerosol formulation or based on the aerosolised formulation. In one aspect caryophyllene oxide and benzyl cinnamate are present in a combined amount of from 0.05 to 4 wt % based on the aerosolisable formulation or based on the aerosol formulation or based on the aerosolised formulation. In one aspect caryophyllene oxide and benzyl cinnamate are present in a combined amount of from 0.08 to 4 wt % based on the aerosolisable formulation or based on the aerosol formulation or based on the aerosolised formulation. In one aspect caryophyllene oxide and benzyl cinnamate are present in a combined amount of no greater than 3 wt % based on the aerosolisable formulation or based on the aerosol formulation or based on the aerosolised formulation. In one aspect caryophyllene oxide and benzyl cinnamate are present in a combined amount of from 0.01 to 3 wt % based on the aerosolisable formulation or based on the aerosol formulation or based on the aerosolised formulation. In one aspect caryophyllene oxide and benzyl cinnamate are present in a combined amount of from 0.02 to 3 wt % based on the aerosolisable formulation or based on the aerosol formulation or based on the aerosolised formulation. In one aspect caryophyllene oxide and benzyl cinnamate are present in a combined amount of from 0.05 to 3 wt % based on the aerosolisable formulation or based on the aerosol formulation or based on the aerosolised formulation. In one aspect caryophyllene oxide and benzyl cinnamate are present in a combined amount of from 0.08 to 3 wt % based on the aerosolisable formulation or based on the aerosol formulation or based on the aerosolised formulation. In one aspect caryophyllene oxide and benzyl cinnamate are present in a combined amount of no greater than 2 wt % based on the aerosolisable formulation or based on the aerosol formulation or 35 based on the aerosolised formulation. In one aspect caryophyllene oxide and benzyl cinnamate are present in a combined amount of from 0.01 to 2 wt % based on the aerosolisable formulation or based on the aerosol formulation or based on the aerosolised formulation. In one aspect caryophyllene oxide and benzyl cinnamate are present in a combined amount of from 0.02 to 2 wt % based on the aerosolisable formulation or based on the aerosol formulation or based on the aerosolised formulation. In one aspect caryophyllene oxide and benzyl cinnamate are present in a combined amount of from 0.05 to 2 wt % based on the aerosolisable formulation or based on the aerosol formulation or based on the aerosolised formulation. In one aspect caryophyllene oxide and benzyl cinnamate are present in a combined amount of from 0.08 to 2 wt % based on the aerosolisable formulation or based on the aerosol formulation or based on the aerosolised formulation. In one aspect caryophyllene oxide and benzyl cinnamate are present in a combined amount of no greater than 1 wt % based on the aerosolisable formulation or based on the aerosol formulation or based on the aerosolised formulation. In one aspect caryophyllene oxide and benzyl cinnamate are present in a combined amount of from 0.01 to 1 wt % based on the aerosolisable formulation or based on the aerosol formulation or based on the aerosolised formulation. In one aspect caryophyllene oxide and benzyl cinnamate are present in a combined amount of from 0.02 to 1 wt % based on the aerosolisable formulation or based on the aerosol formulation or based on the aerosolised formulation. In one aspect caryophyllene oxide and benzyl cinnamate are present in a combined amount of from 0.05 to 1 wt % based on the aerosolisable formulation or based on the aerosol formulation or based on the aerosolised formulation. In one aspect caryophyllene oxide and benzyl cinnamate are present in a combined amount of from 0.08 to 1 wt % based on the aerosolisable formulation or based on the aerosol formulation or based on the aerosolised formulation. In one aspect caryophyllene oxide and benzyl cinnamate are present in a combined amount of from 0.1 to 1 wt % based on the aerosolisable formulation or based on the aerosol formulation or based on the aerosolised formulation.

### Binders

As discussed herein, in one aspect there is provided an aerosolisable formulation comprising
(a) at least one active agent;
(b) aerosol former material;
(c) a modulator selected from a TRPA1 agonist, a TRPM8 agonist, a TRPV1 agonist, a TRPV3 agonist, a TRPA1 antagonist, a TRPA1 inhibitor and combinations thereof; and
(d) one or more binders.

Suitably, the aerosolisable formulation comprises from about 10 wt % to about 80 wt % binder(s), for example from about 10 wt %, 15 wt %, 20 wt %, or 25 wt % to about 70 wt %, 60 wt %, 50 wt %, 45 wt %, 40 wt %, 35 wt % or 30 wt % of one or more binders (all calculated on a dry weight basis). For example, the aerosolisable formulation may comprise about 10-50 wt %, 15-45 wt %, 20-45 wt %, 15-40 wt %, or 20-40 wt % of the binder(s).

In some embodiments, the aerosolisable formulation may comprise about 20-40 wt % of the binder(s).

In some embodiments, the one or more binders comprises one or more gelling agent(s). In some embodiments, the one or more binders consist of one or more gelling agent(s). In some embodiments, the gelling agent comprises a hydrocolloid.

In some embodiments, the one or more binders comprises (or is) one or more compounds selected from polysaccharide gelling agents, such as alginate, pectin, starch or a derivative thereof, cellulose or a derivative thereof, pullulan, carrageenan, agar and agarose; gelatin; gums, such as xanthan gum, guar gum and acacia gum; silica or silicone compounds, such as PDMS and sodium silicate; clays, such as kaolin; and polyvinyl alcohol.

In some embodiments the one or more binders comprises (or is) one or more polysaccharide gelling agents.

In some embodiments, the polysaccharide gelling agent is selected from alginate, pectin, starch or a derivative thereof, or cellulose or a derivative thereof. In some embodiments the polysaccharide gelling agent is selected from alginate and a cellulose derivative.

In some embodiments, the one or more binders is a polysaccharide gelling agent, optionally wherein the polysaccharide gelling agent is selected from alginate and a cellulose derivative.

In some embodiments, the alginate is sodium alginate.

In some embodiments, the polysaccharide gelling agent is a cellulose derivative. Without wishing to be bound by theory, it is believed that such gelling agents do not react with calcium ions to form crosslinks.

In some embodiments, the polysaccharide gelling agent is alginate.

In some embodiments the binder is not crosslinked. The absence of crosslinks in the gelling agent may facilitate quicker delivery of the cannabinoid(s) (and any optional additional active substances and/or flavours) from the aerosolisable formulation.

Examples of cellulosic binders (also referred to herein as cellulosic gelling agents or cellulose derivatives) include, but are not limited to, microcrystalline cellulose (MCC), hydroxymethyl cellulose, hydroxyethyl cellulose, hydroxypropyl cellulose, carboxymethylcellulose (CMC), hydroxypropyl methylcellulose (HPMC), methyl cellulose, ethyl cellulose, cellulose acetate (CA), cellulose acetate butyrate (CAB), and cellulose acetate propionate (CAP). In some embodiments the cellulose or derivative thereof is selected from microcrystalline cellulose (MCC), hydroxymethyl cellulose, hydroxyethyl cellulose, hydroxypropyl cellulose, carboxymethylcellulose (CMC), hydroxypropyl methylcellulose (HPMC), methyl cellulose, ethyl cellulose, cellulose acetate (CA), cellulose acetate butyrate (CAB), and cellulose acetate propionate (CAP). In some embodiments, the cellulose derivative is MCC and/or CMC.

For example, in some embodiments, the binder comprises (or is) one or more of alginate, pectin, hydroxyethyl cellulose, hydroxypropyl cellulose, carboxymethylcellulose, pullulan, xanthan gum guar gum, carrageenan, agarose, acacia gum, fumed silica, PDMS, sodium silicate, kaolin and polyvinyl alcohol.

In some embodiments, the binder comprises (or is) one or more of hydroxyethyl cellulose, hydroxypropyl cellulose, carboxymethylcellulose, guar gum, acacia gum, alginate and/or pectin.

In some embodiments, the binder comprises (or is) microcrystalline cellulose, carboxymethylcellulose, guar gum, alginate and/or pectin.

In some embodiments, the binder comprises (or is) microcrystalline cellulose, carboxymethylcellulose and guar gum.

In some embodiments, the binder comprises (or is) carboxymethylcellulose and guar gum.

In some embodiments, the binder comprises (or is) carboxymethylcellulose, guar gum and alginate.

In some cases, the binder comprises (or is) alginate and/or pectin, and may be combined with a setting agent (such as a calcium source) during formation of the aerosolisable formulation. In some cases, the aerosolisable formulation may comprise a calcium-crosslinked alginate and/or a calcium-crosslinked pectin.

In some embodiments, the binder comprises (or is) alginate, optionally wherein the alginate is present in the aerosolisable formulation in an amount of from about 1-40 wt %, for example about 5-25 wt %, of the aerosolisable formulation (calculated on a dry weight basis).

In some embodiments, alginate is the only binder present in the aerosolisable formulation.

In other embodiments, the binder comprises alginate and at least one further binder, such as pectin.

In particular embodiments, the binder is carboxymethylcellulose, optionally wherein the carboxymethylcellulose (CMC) is present in an amount of about 1-50 wt %, for example about 5-40 wt % or about 30 wt %. In some embodiments, CMC is the only binder present in the aerosolisable formulation.

### Filler

The aerosolisable formulation may further comprise a filler. Use of a filler may help to reduce tackiness of the aerosolisable formulation, for example if high levels of aerosol-former material are present.

Suitably, the aerosolisable formulation comprises from about 10 wt % to about 60 wt % binder and any optional filler.

In some embodiments, the aerosolisable formulation may comprise less than about 60 wt %, such as less than about 50 wt % of a filler, such as from about 1 wt % to 60 wt %, or 1 wt % to 50 wt %, or 5 wt % to 40 wt %, or 5 wt % to 30 wt %, or 10 wt % to 20 wt %.

In other embodiments, the aerosolisable formulation comprises more than 20 wt %, suitably more than 30 wt % or more than 40 wt % of a filler. In some cases, the aerosolisable formulation comprises about 20 wt % to 60 wt %, or 30 wt % to 60 wt %, or 40 wt % to 60 wt % of a filler.

In other embodiments, the aerosolisable formulation comprises less than 20 wt %, suitably less than 10 wt % or less than 5 wt % of a filler. In some cases, the aerosolisable formulation comprises less than 1 wt % of a filler, and in some cases, comprises no filler.

The filler, if present, may comprise one or more inorganic filler materials, such as calcium carbonate, perlite, vermiculite, diatomaceous earth, colloidal silica, magnesium oxide, magnesium sulphate, magnesium carbonate, and suitable inorganic sorbents, such as molecular sieves. The filler may comprise one or more organic filler materials such as wood pulp; tobacco pulp; hemp fibre; starch and starch derivatives, such as maltodextrin; chitosan; and cellulose and cellulose derivatives, such as ground cellulose, microcrystalline cellulose and nanocrystalline cellulose. In particular cases, the aerosolisable formulation comprises no calcium carbonate such as chalk.

In particular embodiments which include filler, the filler is fibrous. For example, the filler may be a fibrous organic filler material such as wood pulp, tobacco pulp, hemp fibre, cellulose or cellulose derivatives. In some embodiments, the fibrous organic filler material may be wood pulp, hemp fibre, cellulose or cellulose derivatives. In some embodiments, the fibrous filler is wood pulp. Without wishing to be bound by theory, it is believed that including fibrous filler in an aerosolisable formulation may increase the tensile strength of the material. This may be particularly advantageous in examples wherein the aerosolisable formulation is provided as a sheet, such as when an aerosolisable formulation sheet circumscribes a rod of aerosolisable material.

In some embodiments the binder is CMC and is used together with wood pulp as a filler.

In some embodiments the binder is CMC and is used together with wood pulp and cellulose or cellulose derivatives as a filler.

In some embodiments the binder is CMC and guar gum, which is used together with wood pulp and cellulose or cellulose derivatives as a filler.

In some embodiments the binder is CMC, MCC and guar gum, which is used together with wood pulp as a filler.

In some embodiments the binder is alginate and pectin, which is used together with wood pulp as a filler.

In some embodiments, the aerosol-generating composition may further comprise one or more other functional material(s).

In some embodiments, the aerosolisable formulation may further comprise one or more additional active substances and/or flavours, and optionally one or more other functional material.

### Aerosol Generating Composition

In one aspect there is provided an aerosol generating composition comprising
(a) botanical material;
(b) an aerosol former; and
(c) a modulator selected from selected from a TRPA1 agonist, a TRPM8 agonist, a TRPV1 agonist, a TRPV3 agonist, a TRPA1 antagonist, a TRPA1 inhibitor and combinations thereof.

As used herein, the term “botanical” includes any material derived from plants including, but not limited to, extracts, leaves, bark, fibres, stems, roots, seeds, flowers, fruits, pollen, husk, shells or the like. Alternatively, the material may comprise an active compound naturally existing in a botanical, obtained synthetically. The material may be in the form of liquid, gas, solid, powder, dust, crushed particles, granules, pellets, shreds, strips, sheets, or the like. Example botanicals are tobacco, eucalyptus, star anise, hemp, cocoa, cannabis, fennel, lemongrass, peppermint, spearmint, rooibos, chamomile, flax, ginger, Ginkgo biloba, hazel, hibiscus, laurel, licorice (liquorice), matcha, mate, orange skin, papaya, rose, sage, tea such as green tea or black tea, thyme, clove, cinnamon, coffee, aniseed (anise), basil, bay leaves, cardamom, coriander, cumin, nutmeg, oregano, paprika, rosemary, saffron, lavender, lemon peel, mint, juniper, elderflower, vanilla, wintergreen, beefsteak plant, curcuma, turmeric, sandalwood, cilantro, bergamot, orange blossom, myrtle, cassis, valerian, pimento, mace, damien, marjoram, olive, lemon balm, lemon basil, chive, carvi, verbena, tarragon, geranium, mulberry, ginseng, theanine, theacrine, maca, ashwagandha, damiana, guarana, chlorophyll, baobab or any combination thereof. The mint may be chosen from the following mint varieties: Mentha Arventis, Mentha c.v., Mentha niliaca, Mentha piperita, Mentha piperita citrata c.v., Mentha piperita c.v, Mentha spicata crispa, Mentha cardifolia, Memtha longifolia, Mentha suaveolens variegata, Mentha pulegium, Mentha spicata c.v. and Mentha suaveolens.

In some embodiments, the botanical comprises tobacco. In some embodiments, the botanical is tobacco. In some embodiments, the botanical is selected from eucalyptus, star anise, cocoa and hemp. In some embodiments, the botanical comprises rooibos and/or fennel. In some embodiments, the botanical is selected from rooibos and fennel. In some embodiments, the botanical comprises rooibos.

In one aspect there is provided a consumable for use in a non-combustible aerosol provision device, the consumable comprising an aerosolisable formulation composition as defined herein.

In one aspect there is provided a non-combustible aerosol provision system comprising the consumable of the present invention and a non-combustible aerosol provision device, the non-combustible aerosol provision device comprising an aerosol-generation device arranged to generate aerosol from the consumable when the consumable is used with the non-combustible aerosol provision device.

In one aspect there is provided use of an aerosolisable formulation composition as defined herein in a consumable for use with a non-combustible aerosol provision device, the non-combustible aerosol provision device comprising an aerosol-generation device arranged to generate aerosol from the consumable when the consumable is used with the non-combustible aerosol provision device.

### Aerosol-Former Material

The aerosol-former material may comprise one or more constituents capable of forming an aerosol.

Suitably, the aerosolisable formulation may comprise from about 5 wt % to about 30 wt % of aerosol-former material (calculated on a dry weight basis), for example about 7 wt %, 10 wt % or 12 wt % to about 15 wt %, 17 wt %, 20 wt % or 25 wt %. In some embodiments the aerosolisable formulation may comprise about 5 to 25 wt % aerosol-former material. In some embodiments, the aerosolisable formulation may comprise about 5 to 20 wt %, such as about 5 to 15 wt % aerosol-former material. In some embodiments the aerosolisable formulation may comprise about 10 to 20 wt % aerosol-former material, such as about 10 to 15 wt % aerosol-former material.

In some embodiments, the aerosol-former material may comprise one or more of glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

In some embodiments, the aerosol-former material may comprise one or more of erythritol, propylene glycol, glycerol, and triacetin. In some cases, the aerosol-former material comprises, consists essentially of or consists of one or more of propylene glycol, glycerol, triethyl citrate and benzyl alcohol. In some cases, the aerosol-former material comprises, consists essentially of or consists of glycerol, or a mixture of glycerol and propylene glycol.

In some embodiments, the aerosol-former material comprises a mixture of glycerol and propylene glycol in a weight ratio of glycerol to propylene glycol of about 3:1 to 1:3, about 2:1 to 1:2, about 1.5:1 to 1:1.5, about 55:45 to 45:55, or about 45:55.

The aerosol-former material may act as a plasticiser. If the content of the plasticiser is too high, the aerosolisable formulation may absorb water resulting in a material that does not create an appropriate consumption experience in use. If the plasticiser content is too low, the aerosolisable formulation may be brittle and easily broken. The plasticiser content specified herein provides an aerosolisable formulation flexibility which allows a sheet of the aerosolisable formulation or aerosol-generating composition to be wound onto a bobbin, which is useful in manufacture of aerosol generating articles (consumables).

### Active Agent

The aerosolisable formulation, aerosol formulation and the aerosolised formulation comprise an active agent. By “active agent” it is meant an agent which has a biological effect, such as sensorial and/or physiological effect, on a subject when the aerosol is inhaled. In one aspect by “active agent” it is meant an agent which has a physiological effect on a subject when the aerosol is inhaled. In some cases, the active may be a flavour. The active agent may for example be selected from nutraceuticals, nootropics, psychoactives and sensates. The active substance may be naturally occurring or synthetically obtained. The active agent may comprise for example nicotine, caffeine, taurine, theine, vitamins such as B6 or B12 or C, melatonin, cannabinoids, or constituents, derivatives, or combinations thereof. The active agent may comprise one or more constituents, derivatives or extracts of tobacco, cannabis or another botanical. In some embodiments, the active agent comprises caffeine, melatonin or vitamin B12. The one or more active agents may be selected from nicotine, botanicals, and mixtures thereof. The one or more active agents may be of synthetic or natural origin. The active could be an extract from a botanical, such as from a plant in the tobacco family. In one aspect the active agent is at least nicotine. In one aspect the active agent consists of nicotine and/or salts thereof.

As noted herein, the active agent may comprise one or more constituents, derivatives or extracts of cannabis, such as one or more cannabinoids or terpenes. Cannabinoids are a class of natural or synthetic chemical compounds that act on cannabinoid receptors (i.e., CB1 and CB2) in cells that repress neurotransmitter release in the brain. Cannabinoids are cyclic molecules exhibiting particular properties such as the ability to cross the blood-brain barrier with ease. Cannabinoids may be naturally occurring (phytocannabinoids) from plants such as cannabis, (endocannabinoids) from animals, or artificially manufactured (synthetic cannabinoids). Cannabis species express at least 85 different phytocannabinoids, and these may be divided into subclasses, including cannabigerols, cannabichromenes, cannabidiols, tetrahydrocannabinols, cannabinols and cannabinodiols, and other cannabinoids, such as cannabigerol (CBG), cannabichromene (CBC), cannabidiol (CBD), tetrahydrocannabinol (THC), including its isomers Δ^{6a,10a}-tetrahydrocannabinol (Δ^{6a,10a}-THC), Δ^{6a(7)}-tetrahydrocannabinol (Δ^{6a(7)}-THC), Δ⁸-tetrahydrocannabinol (Δ⁸-THC), Δ⁹-tetrahydrocannabinol (Δ⁹-THC), Δ¹⁰-tetrahydrocannabinol (Δ¹⁰-THC), Δ^{9,11}-tetrahydrocannabinol (Δ^{9,11}-THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), and tetrahydrocannabivarinic acid (THCV A).

Although the legal status of specific cannabinoids varies from jurisdiction to jurisdiction, certain active components, for example cannabidiol (CBD), tetrahydrocannabinol (THC) and cannabinol (CBN), are being considered for use in a wide variety of applications, such as in formulations for use in aerosol delivery systems. However, the stability of cannabinoids, such as cannabidiol (CBD), tetrahydrocannabinol (THC) and cannabinol (CBN), has been found to vary depending on certain environmental conditions, such as exposure to air or light, or variation in temperature and pH. This may have unintended and detrimental consequences.

For example, CBD may oxidise and degrade when exposed to light and/or air to form cannabidiol hydroxyquinone (CBDHQ or HU-331) and its isomeric or functional derivatives. Furthermore, CBD may be converted to Δ⁹-tetrahydrocannabinol (Δ⁹-THC) in response to variations in temperature and/or pH. As a result, the accuracy of the specified cannabinoid content and/or concentration may vary widely in the formulations, while regulated and restricted cannabinoids may be produced unintentionally that will render the product as illicit or unlicensed in certain jurisdictions. As such, there is a desire to provide formulations comprising one or more cannabinoids that maintain a high degree of purity during manufacture and storage, and in turn prevent the loss or degradation of one or more cannabinoids, such as cannabidiol (CBD), tetrahydrocannabinol (THC) or cannabinol (CBN), in a formulation.

In addition to these stability requirements for cannabinoid formulations, consumer feedback on CBD formulations used in aerosol delivery systems, otherwise referred to as CBD aerosolisable materials, has in some instances been negative with regards to sensorial experience. The natural taste of CBD is often described as ‘earthy’ or ‘grassy’, and whilst the addition of one or more flavouring agents has been previously used in an attempt to mask this taste, this has not always been successful. In particular, consumers have indicated that flavoured CBD aerosolisable materials still can have bitter taste or off notes associated with the taste such that the inhalation of the CBD aerosol may be unpleasant and unsatisfactory. Users may even decide to avoid CBD aerosolisable materials all together; being put off by the taste, aroma and/or other sensation experienced during use. It is known that negative sensorial experiences associated with CBD may originate from TRPA1 agonist activation. As such, antagonists and/or inhibitors for TRPA1 may be suitable for reducing the negative physiological experiences associated with aerosolisable materials comprising CBD. This knowledge can be extended to other cannabinoids. The modulators of the present invention may improve these negative sensorial experiences.

In one embodiment, the cannabinoid is a synthetic cannabinoid. In one embodiment, the cannabinoid is added to the material in the form of an isolate. An isolate is an extract from a plant, such as a cannabis plant. The cannabinoid(s) of interest are typically present in a high degree of purity, for example greater than 95%, greater than 96%, greater than 97%, greater than 98%, or around 99% purity. A synthetic cannabinoid is one which has been derived from a chemical synthesis as opposed to being isolated from a plant or biological source.

In one embodiment the cannabinoid(s) of interest are selected from cannabigerol (CBG), cannabichromene (CBC), cannabidiol (CBD), tetrahydrocannabinol (THC), including its isomers Δ^{6a,10a}-tetrahydrocannabinol (Δ^{6a,10a}-THC), Δ^{6a(7)}-tetrahydrocannabinol (Δ^{6a(7)}-THC), Δ⁸-tetrahydrocannabinol (Δ⁸-THC), Δ⁹-tetrahydrocannabinol (Δ⁹-THC), Δ¹⁰-tetrahydrocannabinol (Δ¹⁰-THC), Δ^{9,11}-tetrahydrocannabinol (Δ^{9,11}-THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), and tetrahydrocannabivarinic acid (THCV A). In one embodiment the cannabinoid(s) of interest are selected from cannabigerol (CBG), cannabichromene (CBC), cannabidiol (CBD), Δ⁸-tetrahydrocannabinol (Δ⁸-THC), Δ⁹-tetrahydrocannabinol (Δ⁹-THC) and cannabinol (CBN).

In one embodiment the cannabinoid(s) of interest are selected from cannabidiol (CBD), Δ⁸-tetrahydrocannabinol (Δ⁸-THC), Δ⁹-tetrahydrocannabinol (Δ⁹-THC). In one embodiment the cannabinoid of interest is cannabidiol (CBD). In one embodiment the cannabinoid of interest is Δ⁸-tetrahydrocannabinol (Δ⁸-THC). In one embodiment the cannabinoid of interest is Δ⁹-tetrahydrocannabinol (Δ⁹-THC). In one embodiment the cannabinoid of interest is cannabinol (CBN).

In one embodiment the cannabinoid(s) of interest are selected from cannabidiol (CBD), Δ⁸-tetrahydrocannabinol (Δ⁸-THC), Δ⁹-tetrahydrocannabinol (Δ⁹-THC), cannabinol (CBN), cannabigerol (CBG), cannabichromene (CBC), and mixtures thereof. In one embodiment the cannabinoid of interest is at least cannabidiol (CBD). In one embodiment the cannabinoid of interest is at least Δ⁸-tetrahydrocannabinol (Δ⁸-THC). In one embodiment the cannabinoid of interest is at least Δ⁹-tetrahydrocannabinol (Δ⁹-THC). In one embodiment the cannabinoid of interest is at least cannabinol (CBN). In one embodiment the cannabinoid of interest is at least cannabigerol (CBG). In one embodiment the cannabinoid of interest is at least cannabichromene (CBC), and mixtures thereof.

The cannabinoid may be present in the formulation based on a mg/ml basis of the formulation.

In one embodiment, the cannabinoid is present in an amount of from about 5 mg/ml up to about 300 mg/ml. In one embodiment, the cannabinoid is present in an amount of from about 5 mg/ml up to about 250 mg/ml. In one embodiment, the cannabinoid is present in an amount of from about 5 mg/ml up to about 200 mg/ml. In one embodiment, the cannabinoid is present in an amount of from about 5 mg/ml up to about 150 mg/ml. In one embodiment, the cannabinoid is present in an amount of from about 5 mg/ml up to about 100 mg/ml. In one embodiment, the cannabinoid is present in an amount of from about 5 mg/ml up to about 90 mg/ml. In one embodiment, the cannabinoid is present in an amount of from about 5 mg/ml up to about 80 mg/ml. In one embodiment, the cannabinoid is present in an amount of from about 5 mg/ml up to about 70 mg/ml. In one embodiment, the cannabinoid is present in an amount of from about 5 mg/ml up to about 60 mg/ml. In one embodiment, the cannabinoid is present in an amount of from about 5 mg/ml up to about 50 mg/ml. In one embodiment, the cannabinoid is present in an amount of from about 5 mg/ml up to about 40 mg/ml. In one embodiment, the cannabinoid is present in an amount of from about 5 mg/ml up to about 30 mg/ml. In one embodiment, the cannabinoid is present in an amount of from about 5 mg/ml up to about 20 mg/ml. In one embodiment, the cannabinoid is present in an amount of from about 5 mg/ml up to about 10 mg/ml.

In one embodiment, the cannabinoid is present in an amount of about 5 mg/ml or more. In one embodiment, the cannabinoid is present in an amount of about 10 mg/ml or more. In one embodiment, the cannabinoid is present in an amount of about 15 mg/ml or more. In one embodiment, the cannabinoid is present in an amount of about 20 mg/ml or more. In one embodiment, the cannabinoid is present in an amount of about 25 mg/ml or more. In one embodiment, the cannabinoid is present in an amount of about 30 mg/ml or more. In one embodiment, the cannabinoid is present in an amount of about 35 mg/ml or more. In one embodiment, the cannabinoid is present in an amount of about 40 mg/ml or more. In one embodiment, the cannabinoid is present in an amount of about 45 mg/ml or more. In one embodiment, the cannabinoid is present in an amount of about 50 mg/ml or more. In one embodiment, the cannabinoid is present in an amount of about 55 mg/ml or more. In one embodiment, the cannabinoid is present in an amount of about 60 mg/ml or more. In one embodiment, the cannabinoid is present in an amount of about 65 mg/ml or more. In one embodiment, the cannabinoid is present in an amount of about 70 mg/ml or more. In one embodiment, the cannabinoid is present in an amount of about 80 mg/ml or more. In one embodiment, the cannabinoid is present in an amount of about 90 mg/ml or more.

As noted herein, the active agent may comprise or be derived from one or more botanicals or constituents, derivatives or extracts thereof.

In some embodiments, the active agent comprises or derived from one or more botanicals or constituents, derivatives or extracts thereof and the botanical is tobacco.

In some embodiments, the active agent comprises or derived from one or more botanicals or constituents, derivatives or extracts thereof and the botanical is selected from eucalyptus, star anise, cocoa and hemp.

In some embodiments, the active agent comprises or derived from one or more botanicals or constituents, derivatives or extracts thereof and the botanical is selected from rooibos and fennel.

The aerosolisable material includes one or more sensates at a specified concentration. By the term “sensate compound” or “sensate”-used interchangeably herein—is meant a compound that triggers a sensation mediated by the trigeminal nerve of a user. The use of sensate compounds is well-documented in the food and pharmaceutical industry, and the triggered sensations include cooling, warming, and tingling sensations. When used in an aerosolisable material, such sensations should be experienced in the oral cavity, the nasal cavity and/or by the skin of the user. The present disclosure is not limited in this respect. In one embodiment, the one or more sensates are selected from cooling agents, warming agents or tingling agents. The terms “cooling”, “warming” and “tingling” are well-understood in the art.

Cooling agents, warming agents and tingling agents are each typically small organic molecules which deliver a cooling, warming or tingling sensation to a user upon contact with the oral cavity, nasal cavity and/or skin. This sensation falls under the category of chemesthetic sensations and arises because the small organic molecule activates certain receptors in the skin and/or mucous membranes. The experience of a cooling, warming and/or tingling sensation thus relies on chemesthesis of the user. Chemesthesis is also referred to in the art as the “common chemical sense” or trigeminal chemosensation because it typically refers to sensations that are mediated by the trigeminal nerve and which are elements of the somatosensory system, distinguishing them from olfaction (sense of smell) and taste.

In one embodiment, the one or more sensates comprise a cooling agent. The cooling agent is typically not menthol. In one embodiment, the one or more sensates comprise a cooling agent which is selected from the group consisting of: , N-ethyl-5-methyl-2-(propan-2-yl) cyclohexanecarboxamide,, ethyl-2-(5-methyl-2-propan-2-yl cyclohexanecarbonyl amino) acetate,, N-(4-methoxyphenyl)-p-menthanecarboxamide,, N-2,3-trimethyl-2-propan-2-yl butanamide,, N-(2-pyridin-2-yl)ethyl)menthyl carboxamide,, menthone-1,2-glycerol ketal,, menthyl lactate,, isopulegol, 3-menthoxypropan-1,2-diol, and menthyl succinate.

In one embodiment, the one or more sensates comprise a warming agent or a tingling agent. In one embodiment, the warming agent or tingling agent is selected from the group consisting of vanilloids, sanshools, piperine, allyl isothiocyanate, cinnamyl phenylpropyl compounds, ethyl esters, and combinations thereof, or the warming agent or tingling agent is an extract from at least one of horseradish oil, ginger oil, black pepper, long pepper, Szechuan pepper, cayenne pepper, Uzazi or mustard oil.

In one embodiment the one or more sensates consist of cooling agents. In one embodiment the one or more sensates consist of warming agents. In one embodiment the one or more sensates consist of tingling agents. In one embodiment the one or more sensates consist of cooling agents and warming agents. In one embodiment the one or more sensates consist of cooling agents and tingling agents. In one embodiment the one or more sensates consist of warming agents and tingling agents.

In one aspect the one or more active agents are present in a combined amount (such as nicotine is present in an amount) of no greater than 50 wt % based on the aerosolisable formulation or based on the aerosol formulation or based on the aerosolised formulation. In one aspect the one or more active agents are present in a combined amount (such as nicotine is present in an amount) of from 0.01 to 50 wt % based on the aerosolisable formulation or based on the aerosol formulation or based on the aerosolised formulation. In one aspect the one or more active agents are present in a combined amount (such as nicotine is present in an amount) of no greater than 40 wt % based on the aerosolisable formulation or based on the aerosol formulation or based on the aerosolised formulation. In one aspect the one or more active agents are present in a combined amount (such as nicotine is present in an amount) of from 0.01 to 40 wt % based on the aerosolisable formulation or based on the aerosol formulation or based on the aerosolised formulation. In one aspect the one or more active agents are present in a combined amount (such as nicotine is present in an amount) of no greater than 30 wt % based on the aerosolisable formulation or based on the aerosol formulation or based on the aerosolised formulation. In one aspect the one or more active agents are present in a combined amount (such as nicotine is present in an amount) of from 0.01 to 30 wt % based on the aerosolisable formulation or based on the aerosol formulation or based on the aerosolised formulation. In one aspect the one or more active agents are present in a combined amount (such as nicotine is present in an amount) of no greater than 20 wt % based on the aerosolisable formulation or based on the aerosol formulation or based on the aerosolised formulation. In one aspect the one or more active agents are present in a combined amount (such as nicotine is present in an amount) of from 0.01 to 20 wt % based on the aerosolisable formulation or based on the aerosol formulation or based on the aerosolised formulation. In one aspect the one or more active agents are present in a combined amount (such as nicotine is present in an amount) of no greater than 10 wt % based on the aerosolisable formulation or based on the aerosol formulation or based on the aerosolised formulation. In one aspect the one or more active agents are present in a combined amount (such as nicotine is present in an amount) of from 0.01 to 10 wt % based on the aerosolisable formulation or based on the aerosol formulation or based on the aerosolised formulation. In one aspect the one or more active agents are present in a combined amount (such as nicotine is present in an amount) of no greater than 8 wt % based on the aerosolisable formulation or based on the aerosol formulation or based on the aerosolised formulation. In one aspect the one or more active agents are present in a combined amount (such as nicotine is present in an amount) of from 0.01 to 8 wt % based on the aerosolisable formulation or based on the aerosol formulation or based on the aerosolised formulation. In one aspect the one or more active agents are present in a combined amount (such as nicotine is present in an amount) of no greater than 6 wt % based on the aerosolisable formulation or based on the aerosol formulation or based on the aerosolised formulation. In one aspect the one or more active agents are present in a combined amount (such as nicotine is present in an amount) of from 0.01 to 6 wt % based on the aerosolisable formulation or based on the aerosol formulation or based on the aerosolised formulation. In one aspect the one or more active agents are present in a combined amount (such as nicotine is present in an amount) of from 0.02 to 6 wt % based on the aerosolisable formulation or based on the aerosol formulation or based on the aerosolised formulation. In one aspect the one or more active agents are present in a combined amount (such as nicotine is present in an amount) of from 0.05 to 6 wt % based on the aerosolisable formulation or based on the aerosol formulation or based on the aerosolised formulation. In one aspect the one or more active agents are present in a combined amount (such as nicotine is present in an amount) of from 0.08 to 6 wt % based on the aerosolisable formulation or based on the aerosol formulation or based on the aerosolised formulation. In one aspect the one or more active agents are present in a combined amount (such as nicotine is present in an amount) of from 0.01 to 5 wt % based on the aerosolisable formulation or based on the aerosol formulation or based on the aerosolised formulation. In one aspect the one or more active agents are present in a combined amount (such as nicotine is present in an amount) of from 0.02 to 5 wt % based on the aerosolisable formulation or based on the aerosol formulation or based on the aerosolised formulation. In one aspect the one or more active agents are present in a combined amount (such as nicotine is present in an amount) of from 0.05 to 5 wt % based on the aerosolisable formulation or based on the aerosol formulation or based on the aerosolised formulation. In one aspect the one or more active agents are present in a combined amount (such as nicotine is present in an amount) of from 0.08 to 5 wt % based on the aerosolisable formulation or based on the aerosol formulation or based on the aerosolised formulation. In one aspect the one or more active agents are present in a combined amount (such as nicotine is present in an amount) of no greater than 4 wt % based on the aerosolisable formulation or based on the aerosol formulation or based on the aerosolised formulation. In one aspect the one or more active agents are present in a combined amount (such as nicotine is present in an amount) of from 0.01 to 4 wt % based on the aerosolisable formulation or based on the aerosol formulation or based on the aerosolised formulation. In one aspect the one or more active agents are present in a combined amount (such as nicotine is present in an amount) of from 0.02 to 4 wt % based on the aerosolisable formulation or based on the aerosol formulation or based on the aerosolised formulation. In one aspect the one or more active agents are present in a combined amount (such as nicotine is present in an amount) of from 0.05 to 4 wt % based on the aerosolisable formulation or based on the aerosol formulation or based on the aerosolised formulation. In one aspect the one or more active agents are present in a combined amount (such as nicotine is present in an amount) of from 0.08 to 4 wt % based on the aerosolisable formulation or based on the aerosol formulation or based on the aerosolised formulation. In one aspect the one or more active agents are present in a combined amount (such as nicotine is present in an amount) of no greater than 3 wt % based on the aerosolisable formulation or based on the aerosol formulation or based on the aerosolised formulation. In one aspect the one or more active agents are present in a combined amount (such as nicotine is present in an amount) of from 0.01 to 3 wt % based on the aerosolisable formulation or based on the aerosol formulation or based on the aerosolised formulation. In one aspect the one or more active agents are present in a combined amount (such as nicotine is present in an amount) of from 0.02 to 3 wt % based on the aerosolisable formulation or based on the aerosol formulation or based on the aerosolised formulation. In one aspect the one or more active agents are present in a combined amount (such as nicotine is present in an amount) of from 0.05 to 3 wt % based on the aerosolisable formulation or based on the aerosol formulation or based on the aerosolised formulation. In one aspect the one or more active agents are present in a combined amount (such as nicotine is present in an amount) of from 0.08 to 3 wt % based on the aerosolisable formulation or based on the aerosol formulation or based on the aerosolised formulation. In one aspect the one or more active agents are present in a combined amount (such as nicotine is present in an amount) of no greater than 2 wt % based on the aerosolisable formulation or based on the aerosol formulation or based on the aerosolised formulation. In one aspect the one or more active agents are present in a combined amount (such as nicotine is present in an amount) of from 0.01 to 2 wt % based on the aerosolisable formulation or based on the aerosol formulation or based on the aerosolised formulation. In one aspect the one or more active agents are present in a combined amount (such as nicotine is present in an amount) of from 0.02 to 2 wt % based on the aerosolisable formulation or based on the aerosol formulation or based on the aerosolised formulation. In one aspect the one or more active agents are present in a combined amount (such as nicotine is present in an amount) of from 0.05 to 2 wt % based on the aerosolisable formulation or based on the aerosol formulation or based on the aerosolised formulation. In one aspect the one or more active agents are present in a combined amount (such as nicotine is present in an amount) of from 0.08 to 2 wt % based on the aerosolisable formulation or based on the aerosol formulation or based on the aerosolised formulation. In one aspect the one or more active agents are present in a combined amount (such as nicotine is present in an amount) of no greater than 1 wt % based on the aerosolisable formulation or based on the aerosol formulation or based on the aerosolised formulation. In one aspect the one or more active agents are present in a combined amount (such as nicotine is present in an amount) of from 0.01 to 1 wt % based on the aerosolisable formulation or based on the aerosol formulation or based on the aerosolised formulation. In one aspect the one or more active agents are present in a combined amount (such as nicotine is present in an amount) of from 0.02 to 1 wt % based on the aerosolisable formulation or based on the aerosol formulation or based on the aerosolised formulation. In one aspect the one or more active agents are present in a combined amount (such as nicotine is present in an amount) of from 0.05 to 1 wt % based on the aerosolisable formulation or based on the aerosol formulation or based on the aerosolised formulation. In one aspect the one or more active agents are present in a combined amount (such as nicotine is present in an amount) of from 0.08 to 1 wt % based on the aerosolisable formulation or based on the aerosol formulation or based on the aerosolised formulation. In one aspect the one or more active agents are present in a combined amount (such as nicotine is present in an amount) of from 0.1 to 1 wt % based on the aerosolisable formulation or based on the aerosol formulation or based on the aerosolised formulation. In one aspect the one or more active agents are present in a combined amount (such as nicotine is present in an amount) of no greater than 0.6 wt % based on the aerosolisable formulation or based on the aerosol formulation or based on the aerosolised formulation. In one aspect the one or more active agents are present in a combined amount (such as nicotine is present in an amount) of from 0.01 to 0.6 wt % based on the aerosolisable formulation or based on the aerosol formulation or based on the aerosolised formulation. In one aspect the one or more active agents are present in a combined amount (such as nicotine is present in an amount) of from 0.02 to 0.6 wt % based on the aerosolisable formulation or based on the aerosol formulation or based on the aerosolised formulation. In one aspect the one or more active agents are present in a combined amount (such as nicotine is present in an amount) of from 0.05 to 0.6 wt % based on the aerosolisable formulation or based on the aerosol formulation or based on the aerosolised formulation. In one aspect the one or more active agents are present in a combined amount (such as nicotine is present in an amount) of from 0.08 to 0.6 wt % based on the aerosolisable formulation or based on the aerosol formulation or based on the aerosolised formulation. In one aspect the one or more active agents are present in a combined amount (such as nicotine is present in an amount) of from 0.1 to 0.6 wt % based on the aerosolisable formulation or based on the aerosol formulation or based on the aerosolised formulation. In one aspect the one or more active agents are present in a combined amount (such as nicotine is present in an amount) of no greater than 0.5 wt % based on the aerosolisable formulation or based on the aerosol formulation or based on the aerosolised formulation. In one aspect the one or more active agents are present in a combined amount (such as nicotine is present in an amount) of from 0.01 to 0.5 wt % based on the aerosolisable formulation or based on the aerosol formulation or based on the aerosolised formulation. In one aspect the one or more active agents are present in a combined amount (such as nicotine is present in an amount) of from 0.02 to 0.5 wt % based on the aerosolisable formulation or based on the aerosol formulation or based on the aerosolised formulation. In one aspect the one or more active agents are present in a combined amount (such as nicotine is present in an amount) of from 0.05 to 0.5 wt % based on the aerosolisable formulation or based on the aerosol formulation or based on the aerosolised formulation. In one aspect the one or more active agents are present in a combined amount (such as nicotine is present in an amount) of from 0.08 to 0.5 wt % based on the aerosolisable formulation or based on the aerosol formulation or based on the aerosolised formulation. In one aspect the one or more active agents are present in a combined amount (such as nicotine is present in an amount) of no greater than 0.2 wt % based on the aerosolisable formulation or based on the aerosol formulation or based on the aerosolised formulation. In one aspect the one or more active agents are present in a combined amount (such as nicotine is present in an amount) of from 0.01 to 0.2 wt % based on the aerosolisable formulation or based on the aerosol formulation or based on the aerosolised formulation. In one aspect the one or more active agents are present in a combined amount (such as nicotine is present in an amount) of from 0.02 to 0.2 wt % based on the aerosolisable formulation or based on the aerosol formulation or based on the aerosolised formulation. In one aspect the one or more active agents are present in a combined amount (such as nicotine is present in an amount) of from 0.05 to 0.2 wt % based on the aerosolisable formulation or based on the aerosol formulation or based on the aerosolised formulation. In one aspect the one or more active agents are present in a combined amount (such as nicotine is present in an amount) of from 0.08 to 0.2 wt % based on the aerosolisable formulation or based on the aerosol formulation or based on the aerosolised formulation. In one aspect the one or more active agents are present in a combined amount (such as nicotine is present in an amount) of no greater than 0.1 wt % based on the aerosolisable formulation or based on the aerosol formulation or based on the aerosolised formulation. In one aspect the one or more active agents are present in a combined amount (such as nicotine is present in an amount) of from 0.01 to 0.1 wt % based on the aerosolisable formulation or based on the aerosol formulation or based on the aerosolised formulation. In one aspect the one or more active agents are present in a combined amount (such as nicotine is present in an amount) of from 0.02 to 0.1 wt % based on the aerosolisable formulation or based on the aerosol formulation or based on the aerosolised formulation. In one aspect the one or more active agents are present in a combined amount (such as nicotine is present in an amount) of from 0.05 to 0.1 wt % based on the aerosolisable formulation or based on the aerosol formulation or based on the aerosolised formulation. In one aspect the one or more active agents are present in a combined amount (such as nicotine is present in an amount) of from 0.08 to 0.1 wt % based on the aerosolisable formulation or based on the aerosol formulation or based on the aerosolised formulation.

In one aspect cannabinoids are present in an amount of no greater than 50 wt % based on the aerosolisable formulation or based on the aerosol formulation or based on the aerosolised formulation. In one aspect cannabinoids are present in an amount of from 10 to 50 wt % based on the aerosolisable formulation or based on the aerosol formulation or based on the aerosolised formulation. In one aspect cannabinoids are present in an amount of no greater than 40 wt % based on the aerosolisable formulation or based on the aerosol formulation or based on the aerosolised formulation. In one aspect cannabinoids are present in an amount of from 10 to 40 wt % based on the aerosolisable formulation or based on the aerosol formulation or based on the aerosolised formulation. In one aspect cannabinoids are present in an amount of no greater than 30 wt % based on the aerosolisable formulation or based on the aerosol formulation or based on the aerosolised formulation. In one aspect cannabinoids are present in an amount of from 10 to 30 wt % based on the aerosolisable formulation or based on the aerosol formulation or based on the aerosolised formulation. In one aspect cannabinoids are present in an amount of no greater than 20 wt % based on the aerosolisable formulation or based on the aerosol formulation or based on the aerosolised formulation. In one aspect cannabinoids are present in an amount of from 10 to 20 wt % based on the aerosolisable formulation or based on the aerosol formulation or based on the aerosolised formulation. In one aspect cannabinoids are present in an amount of no greater than 15 wt % based on the aerosolisable formulation or based on the aerosol formulation or based on the aerosolised formulation. In one aspect cannabinoids are present in an amount of from 10 to 15 wt % based on the aerosolisable formulation or based on the aerosol formulation or based on the aerosolised formulation.

### Carrier

The carrier comprises one or more constituents capable of forming an aerosol, particularly when evaporated and allowed to condense. Typically, the carrier selected from water, propylene glycol, glycerol and mixtures thereof. However, other suitable carriers may also be used. Therefore, in one aspect there is provided an aerosolisable formulation or aerosol formulation comprising
(a) at least one active agent;
(b) a liquid carrier; and
(c) a modulator selected from a TRPA1 agonist, a TRPM8 agonist, a TRPV1 agonist, a TRPV3 agonist, a TRPA1 antagonist, a TRPA1 inhibitor and combinations thereof.

In one aspect, the carrier comprises at least water. In one aspect, the carrier comprises at least propylene glycol. In one aspect, the carrier comprises at least glycerol. In one aspect, the carrier comprises at least water and glycerol. In one aspect, the carrier comprises at least propylene glycol and glycerol. In one aspect, the carrier comprises at least propylene glycol and water. In one aspect, the carrier comprises at least propylene glycol, glycerol and water.

The carrier may be present in any suitable amount based on the total weight of the aerosolisable material. In one aspect, the carrier constituent comprises at least 50 wt. % based on the total weight of the aerosolisable material. In one aspect, the carrier constituent comprises at least 60 wt. % based on the total weight of the aerosolisable material. In one aspect, the carrier constituent comprises at least 70 wt. % based on the total weight of the aerosolisable material. In one aspect, the carrier constituent comprises at least 80 wt. % based on the total weight of the aerosolisable material. In one aspect, the carrier constituent comprises at least 90 wt. % based on the total weight of the aerosolisable material.

In some embodiments, the carrier may further comprise one or more of glycerol, triethylene glycol, tetraethylene glycol, 1,3-butylene glycol, erythritol, meso-erythritol, ethyl laurate, a diethyl suberate, triethyl citrate, triethylene glycol diacetate, triacetin, a diacetin mixture, benzyl benzoate, benzyl phenyl acetate, tributyrin, lauryl acetate, lauric acid, myristic acid, and propylene carbonate.

In one aspect the carrier is selected from propylene glycol, glycerol, triethyl citrate, benzyl alcohol and mixtures thereof.

In one embodiment, the aerosolisable material is a liquid at about 25° C.

### Acid

As discussed above, the aerosolisable formulation, the aerosol formulation and the aerosolised formulation of the present invention may contain one or more further components. These components may be selected depending on the nature of the formulation. In one aspect, the aerosolisable formulation, the aerosol formulation and the aerosolised formulation further comprises an acid

In one aspect the acid is an organic acid. In one aspect the acid is a carboxylic acid. In one aspect the acid is an organic carboxylic acid.

In one aspect the acid is selected from the group consisting of acetic acid, lactic acid, formic acid, citric acid, benzoic acid, pyruvic acid, levulinic acid, succinic acid, tartaric acid, sorbic acid, propionic acid, phenylacetic acid, and mixtures thereof. In one aspect the acid is selected from the group consisting of citric acid, benzoic acid, levulinic acid, lactic acid, sorbic acid, and mixtures thereof. In one aspect the acid is selected from the group consisting of citric acid, benzoic acid, levulinic acid, and mixtures thereof.

In one aspect, the acid is selected from the group consisting of acetic acid, lactic acid, formic acid, citric acid, benzoic acid, pyruvic acid, levulinic acid, succinic acid, tartaric acid, sorbic acid, propionic acid, phenylacetic acid, salicylic acid, malic acid, caffeic acid, fumaric acid, nicotinic acid, o-toluic acid, m-toluic acid, p-toluic acid, capric acid, glucono delta-lactone, gluconic acid, malonic acid, phosphoric acid, 4-hydroxyphenylacetic acid and mixtures thereof.

In one aspect, the acid is selected from the group consisting of salicylic acid, malic acid, citric acid, succinic acid, caffeic acid, fumaric acid, nicotinic acid, o-toluic acid, m-toluic acid, p-toluic acid, capric acid, glucono delta-lactone, pyruvic acid, sorbic acid, levulinic acid, tartaric acid, gluconic acid, malonic acid, phosphoric acid, 4-hydroxyphenylacetic acid and mixtures thereof.

In one aspect the acid is selected from acids having a pka of from 2 to 5. In one aspect the acid is a weak acid. In one aspect the acid is a weak organic acid.

In one aspect the acid has a solubility in water of at least 5 g/L at 20° C. In one aspect the acid has a solubility in water of at least 10 g/L at 20° C. In one aspect the acid has a solubility in water of at least 20 g/L at 20° C. In one aspect the acid has a solubility in water of at least 50 g/L at 20° C. In one aspect the acid has a solubility in water of at least 100 g/L at 20° C. In one aspect the acid has a solubility in water of at least 200 g/L at 20° C. In one aspect the acid has a solubility in water of at least 300 g/L at 20° C. In one aspect the acid has a solubility in water of at least 400 g/L at 20° C. In one aspect the acid has a solubility in water of at least 500 g/L at 20° C. In one aspect the acid has a solubility in water of at least 600 g/L at 20° C. In one aspect the acid has a solubility in water of at least 700 g/L at 20° C. In one aspect the acid has a solubility in water of at least 800 g/L at 20° C. In one aspect the acid has a solubility in water of at least 900 g/L at 20° C. In one aspect the acid has a solubility in water of at least 1000 g/L at 20° C. In one aspect the acid has a solubility in water of at least 1100 g/L at 20° C.

The molar ratio of acid to active agent (such as nicotine) may be selected as desired. In one aspect the molar ratio of acid to active agent (such as nicotine) is from 5:1 to 1:5. In one aspect the molar ratio of acid to active agent (such as nicotine) is from 4:1 to 1:4. In one aspect the molar ratio of acid to active agent (such as nicotine) is from 3:1 to 1:3. In one aspect the molar ratio of acid to active agent (such as nicotine) is from 2:1 to 1:2. In one aspect the molar ratio of acid to active agent (such as nicotine) is from 1.5:1 to 1:1.5. In one aspect the molar ratio of acid to active agent (such as nicotine) is from 1.2:1 to 1:1.2. In one aspect the molar ratio of acid to active agent (such as nicotine) is from 5:1 to 1:1. In one aspect the molar ratio of acid to active agent (such as nicotine) is from 4:1 to 1:1. In one aspect the molar ratio of acid to active agent (such as nicotine) is from 3:1 to 1:1. In one aspect the molar ratio of acid to active agent (such as nicotine) is from 2:1 to 1:1. In one aspect the molar ratio of acid to active agent (such as nicotine) is from 1.5:1 to 1:1. In one aspect the molar ratio of acid to active agent (such as nicotine) is from 1.2:1 to 1:1.

In one aspect the total content of acid present in the formulation is no greater than 5 mole equivalents based on the active agent (such as nicotine). In one aspect the total content of acid present in the formulation is no greater than 4 mole equivalents based on the active agent (such as nicotine). In one aspect the total content of acid present in the formulation is no greater than 3 mole equivalents based on the active agent (such as nicotine). In one aspect the total content of acid present in the formulation is no greater than 2 mole equivalents based on the active agent (such as nicotine). In one aspect the total content of acid present in the formulation is no greater than 1 mole equivalents based on the active agent (such as nicotine).

In one aspect the total content of acid present in the formulation is no less than 0.01 mole equivalents based on the active agent (such as nicotine). In one aspect the total content of acid present in the formulation is no less than 0.05 mole equivalents based on the active agent (such as nicotine). In one aspect the total content of acid present in the formulation is no less than 0.1 mole equivalents based on the active agent (such as nicotine). In one aspect the total content of acid present in the formulation is no less than 0.2 mole equivalents based on the active agent (such as nicotine). In one aspect the total content of acid present in the formulation is no less than 0.3 mole equivalents based on the active agent (such as nicotine). In one aspect the total content of acid present in the formulation is no less than 0.4 mole equivalents based on the active agent (such as nicotine). In one aspect the total content of acid present in the formulation is no less than 0.5 mole equivalents based on the active agent (such as nicotine). In one aspect the total content of acid present in the formulation is no less than 0.7 mole equivalents based on the active agent (such as nicotine).

In one aspect the acid is present in an amount of no greater than 6 wt % based on the aerosolisable formulation or based on the aerosol formulation. In one aspect the acid is present in an amount of from 0.01 to 6 wt % based on the aerosolisable formulation or based on the aerosol formulation. In one aspect the acid is present in an amount of from 0.02 to 6 wt % based on the aerosolisable formulation or based on the aerosol formulation. In one aspect the acid is present in an amount of from 0.05 to 6 wt % based on the aerosolisable formulation or based on the aerosol formulation. In one aspect the acid is present in an amount of from 0.08 to 6 wt % based on the aerosolisable formulation or based on the aerosol formulation. In one aspect the acid is present in an amount of from 0.01 to 5 wt % based on the aerosolisable formulation or based on the aerosol formulation. In one aspect the acid is present in an amount of from 0.02 to 5 wt % based on the aerosolisable formulation or based on the aerosol formulation. In one aspect the acid is present in an amount of from 0.05 to 5 wt % based on the aerosolisable formulation or based on the aerosol formulation. In one aspect the acid is present in an amount of from 0.08 to 5 wt % based on the aerosolisable formulation or based on the aerosol formulation. In one aspect the acid is present in an amount of no greater than 4 wt % based on the aerosolisable formulation or based on the aerosol formulation. In one aspect the acid is present in an amount of from 0.01 to 4 wt % based on the aerosolisable formulation or based on the aerosol formulation. In one aspect the acid is present in an amount of from 0.02 to 4 wt % based on the aerosolisable formulation or based on the aerosol formulation. In one aspect the acid is present in an amount of from 0.05 to 4 wt % based on the aerosolisable formulation or based on the aerosol formulation. In one aspect the acid is present in an amount of from 0.08 to 4 wt % based on the aerosolisable formulation or based on the aerosol formulation. In one aspect the acid is present in an amount of no greater than 3 wt % based on the aerosolisable formulation or based on the aerosol formulation. In one aspect the acid is present in an amount of from 0.01 to 3 wt % based on the aerosolisable formulation or based on the aerosol formulation. In one aspect the acid is present in an amount of from 0.02 to 3 wt % based on the aerosolisable formulation or based on the aerosol formulation. In one aspect the acid is present in an amount of from 0.05 to 3 wt % based on the aerosolisable formulation or based on the aerosol formulation. In one aspect the acid is present in an amount of from 0.08 to 3 wt % based on the aerosolisable formulation or based on the aerosol formulation. In one aspect the acid is present in an amount of no greater than 2 wt % based on the aerosolisable formulation or based on the aerosol formulation. In one aspect the acid is present in an amount of from 0.01 to 2 wt % based on the aerosolisable formulation or based on the aerosol formulation. In one aspect the acid is present in an amount of from 0.02 to 2 wt % based on the aerosolisable formulation or based on the aerosol formulation. In one aspect the acid is present in an amount of from 0.05 to 2 wt % based on the aerosolisable formulation or based on the aerosol formulation. In one aspect the acid is present in an amount of from 0.08 to 2 wt % based on the aerosolisable formulation or based on the aerosol formulation. In one aspect the acid is present in an amount of no greater than 1 wt % based on the aerosolisable formulation or based on the aerosol formulation. In one aspect the acid is present in an amount of from 0.01 to 1 wt % based on the aerosolisable formulation or based on the aerosol formulation. In one aspect the acid is present in an amount of from 0.02 to 1 wt % based on the aerosolisable formulation or based on the aerosol formulation. In one aspect the acid is present in an amount of from 0.05 to 1 wt % based on the aerosolisable formulation or based on the aerosol formulation. In one aspect the acid is present in an amount of from 0.08 to 1 wt % based on the aerosolisable formulation or based on the aerosol formulation. In one aspect the acid is present in an amount of from 0.1 to 1 wt % based on the aerosolisable formulation or based on the aerosol formulation. In one aspect the acid is present in an amount of no greater than 0.6 wt % based on the aerosolisable formulation or based on the aerosol formulation. In one aspect the acid is present in an amount of from 0.01 to 0.6 wt % based on the aerosolisable formulation or based on the aerosol formulation. In one aspect the acid is present in an amount of from 0.02 to 0.6 wt % based on the aerosolisable formulation or based on the aerosol formulation. In one aspect the acid is present in an amount of from 0.05 to 0.6 wt % based on the aerosolisable formulation or based on the aerosol formulation. In one aspect the acid is present in an amount of from 0.08 to 0.6 wt % based on the aerosolisable formulation or based on the aerosol formulation. In one aspect the acid is present in an amount of from 0.1 to 0.6 wt % based on the aerosolisable formulation or based on the aerosol formulation. In one aspect the acid is present in an amount of no greater than 0.5 wt % based on the aerosolisable formulation or based on the aerosol formulation. In one aspect the acid is present in an amount of from 0.01 to 0.5 wt % based on the aerosolisable formulation or based on the aerosol formulation. In one aspect the acid is present in an amount of from 0.02 to 0.5 wt % based on the aerosolisable formulation or based on the aerosol formulation. In one aspect the acid is present in an amount of from 0.05 to 0.5 wt % based on the aerosolisable formulation or based on the aerosol formulation. In one aspect the acid is present in an amount of from 0.08 to 0.5 wt % based on the aerosolisable formulation or based on the aerosol formulation. In one aspect the acid is present in an amount of no greater than 0.2 wt % based on the aerosolisable formulation or based on the aerosol formulation. In one aspect the acid is present in an amount of from 0.01 to 0.2 wt % based on the aerosolisable formulation or based on the aerosol formulation. In one aspect the acid is present in an amount of from 0.02 to 0.2 wt % based on the aerosolisable formulation or based on the aerosol formulation. In one aspect the acid is present in an amount of from 0.05 to 0.2 wt % based on the aerosolisable formulation or based on the aerosol formulation. In one aspect the acid is present in an amount of from 0.08 to 0.2 wt % based on the aerosolisable formulation or based on the aerosol formulation. In one aspect the acid is present in an amount of no greater than 0.1 wt % based on the aerosolisable formulation or based on the aerosol formulation. In one aspect the acid is present in an amount of from 0.01 to 0.1 wt % based on the aerosolisable formulation or based on the aerosol formulation. In one aspect the acid is present in an amount of from 0.02 to 0.1 wt % based on the aerosolisable formulation or based on the aerosol formulation. In one aspect the acid is present in an amount of from 0.05 to 0.1 wt % based on the aerosolisable formulation or based on the aerosol formulation. In one aspect the acid is present in an amount of from 0.08 to 0.1 wt % based on the aerosolisable formulation or based on the aerosol formulation.

The amount of acid and the solubility of the acid may be selected such that a given amount of the acid will dissolve in the water. In one aspect at 20° C. at least 20% of the acid dissolves in the water. In one aspect at 25° C. at least 20% of the acid dissolves in the water. In one aspect at 30° C. at least 20% of the acid dissolves in the water. In one aspect at 20° C. at least 35% of the acid dissolves in the water. In one aspect at 20° C. at least 40% of the acid dissolves in the water. In one aspect at 20° C. at least 45% of the acid dissolves in the water. In one aspect at 20° C. at least 50% of the acid dissolves in the water. In one aspect at 20° C. at least 55% of the acid dissolves in the water.

As is understood by one skilled in the art, nicotine may exist in unprotonated form, monoprotonated form or diprotonated form. The structures of each of these forms are given below.

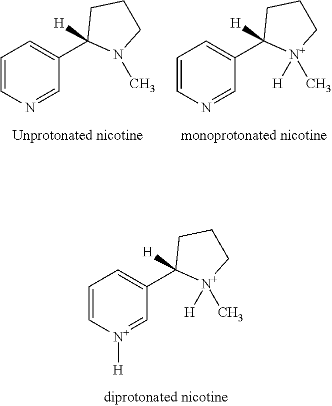

Reference in the specification to protonated form means both monoprotonated nicotine and diprotonated nicotine. Reference in the specification to amounts in the protonated form means the combined amount of monoprotonated nicotine and diprotonated nicotine. Furthermore, when reference is made to a fully protonated formulation it will be understood that at any one time there may be very minor amounts of unprotonated nicotine present, e.g. less than 1% unprotonated.

The formulation may comprise nicotine in protonated form. The formulation may comprise nicotine in unprotonated form. In one aspect the formulation comprises nicotine in unprotonated form and nicotine in monoprotonated form. In one aspect the formulation comprises nicotine in unprotonated form and nicotine in diprotonated form. In one aspect the formulation comprises nicotine in unprotonated form, nicotine in monoprotonated form and nicotine in diprotonated form.

In one aspect at least 5 wt % of the nicotine present in the formulation is in protonated form. In one aspect at least 10 wt % of the nicotine present in the formulation is in protonated form. In one aspect at least 15 wt % of the nicotine present in the formulation is in protonated form. In one aspect at least 20 wt % of the nicotine present in the formulation is in protonated form. In one aspect at least 25 wt % of the nicotine present in the formulation is in protonated form. In one aspect at least 30 wt % of the nicotine present in the formulation is in protonated form. In one aspect at least 35 wt % of the nicotine present in the formulation is in protonated form. In one aspect at least 40 wt % of the nicotine present in the formulation is in protonated form. In one aspect at least 45 wt % of the nicotine present in the formulation is in protonated form. In one aspect at least 50 wt % of the nicotine present in the formulation is in protonated form. In one aspect at least 55 wt % of the nicotine present in the formulation is in protonated form. In one aspect at least 60 wt % of the nicotine present in the formulation is in protonated form. In one aspect at least 65 wt % of the nicotine present in the formulation is in protonated form. In one aspect at least 70 wt % of the nicotine present in the formulation is in protonated form. In one aspect at least 75 wt % of the nicotine present in the formulation is in protonated form. In one aspect at least 80 wt % of the nicotine present in the formulation is in protonated form. In one aspect at least 85 wt % of the nicotine present in the formulation is in protonated form. In one aspect at least 90 wt % of the nicotine present in the formulation is in protonated form. In one aspect at least 95 wt % of the nicotine present in the formulation is in protonated form. In one aspect at least 99 wt % of the nicotine present in the formulation is in protonated form. In one aspect at least 99.9 wt % of the nicotine present in the formulation is in protonated form.

In one aspect from 50 to 95 wt % of the nicotine present in the formulation is in protonated form. In one aspect from 55 to 95 wt % of the nicotine present in the formulation is in protonated form. In one aspect from 60 to 95 wt % of the nicotine present in the formulation is in protonated form. In one aspect from 65 to 95 wt % of the nicotine present in the formulation is in protonated form. In one aspect from 70 to 95 wt % of the nicotine present in the formulation is in protonated form. In one aspect from 75 to 95 wt % of the nicotine present in the formulation is in protonated form. In one aspect from 80 to 95 wt % of the nicotine present in the formulation is in protonated form. In one aspect from 85 to 95 wt % of the nicotine present in the formulation is in protonated form. In one aspect from 90 to 95 wt % of the nicotine present in the formulation is in protonated form.

The relevant amounts of nicotine which are present in the formulation in protonated form are specified herein. These amounts may be readily calculated by one skilled in the art. Nicotine, 3-(1-methylpyrrolidin-2-yl)pyridine, is a diprotic base with pKa of 3.12 for the pyridine ring and 8.02 for the pyrrolidine ring It can exist in pH-dependent protonated (mono- and di-) and non-protonated (free base) forms which have different bioavailability.

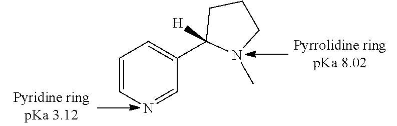

The distribution of protonated and non-protonated nicotine will vary at various pH increments.

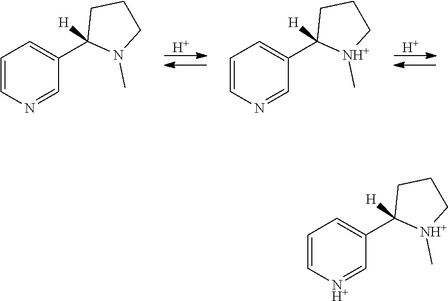

The fraction of non-protonated nicotine will be predominant at high PH levels whilst a decrease in the pH will see an increase of the fraction of protonated nicotine (mono- or di-depending on the pH). If the relative fraction of protonated nicotine and the total amount of nicotine in the sample are known, the absolute amount of protonated nicotine can be calculated.

The relative fraction of protonated nicotine in formulation can be calculated by using the Henderson-Hasselbalch equation, which describes the pH as a derivation of the acid dissociation constant equation, and it is extensively employed in chemical and biological systems. Consider the following equilibrium:

$$B + {H}^{+} ⇄ {BH}^{+}$$

The Henderson-Hasselbalch equation for this equilibrium is:

$$pH = pKa + log \frac{[ B ]}{[ BH + ]}$$

Where [B] is the amount of non-protonated nicotine (i.e. free base), [BH+] the amount of protonated nicotine (i.e. conjugate acid) and pKa is the reference pKa value for the pyrrolidine ring nitrogen of nicotine (pKa=8.02). The relative fraction of protonated nicotine can be derived from the alpha value of the non-protonated nicotine calculated from the Henderson-Hasselbalch equation as:

$$\% protonated nicotine = 100 - \{ \frac{\frac{[ B ]}{[ BH + ]}}{\{ 1 + \frac{[ B ]}{[ BH + ]} \}} * 100 \}$$

Determination of pKa values of nicotine formulations was carried out using the basic approach described in “Spectroscopic investigations into the acid-base properties of nicotine at different temperatures”, Peter M. Clayton, Carl A. Vas, Tam T. T. Bui, Alex F. Drake and Kevin McAdam, .Anal. Methods, 2013,5, 81-88.

### Flavour

The aerosolisable formulation, the aerosol formulation and the aerosolised formulation of the present invention may contain one or more further components. These components may be selected depending on the nature of the formulation. The aerosolisable formulation or the aerosol formulation may comprise one or more flavours or flavouring components. As used herein, the terms “flavour” and “flavourant” refer to materials which, where local regulations permit, may be used to create a desired taste or aroma in a product for adult consumers. They may include extracts (e.g. liquorice, hydrangea, Japanese white bark magnolia leaf, chamomile, fenugreek, clove, menthol, Japanese mint, aniseed, cinnamon, herb, wintergreen, cherry, berry, peach, apple, Drambuie, bourbon, scotch, whiskey, spearmint, peppermint, lavender, cardamom, celery, cascarilla, nutmeg, sandalwood, bergamot, geranium, honey essence, rose oil, vanilla, lemon oil, orange oil, cassia, caraway, cognac, jasmine, ylang-ylang, sage, fennel, piment, ginger, anise, coriander, coffee, or a mint oil from any species of the genus Mentha), flavour enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, oil, liquid, or powder. The one or more flavours may be selected from the group consisting of (4-(para-) methoxyphenyl)-2-butanone, vanillin, y-undecalactone, menthone, 5-propenyl guaethol, menthol, para-mentha-8-thiol-3-one and mixtures thereof. In one aspect the flavour is at least menthol.

If present, the one or more flavours may be present in any suitable amount. In one aspect the one or more flavours are present in a total amount of no greater than 10 wt. % based on the aerosolisable formulation. In one aspect the one or more flavours are present in a total amount of no greater than 7 wt. % based on the aerosolisable formulation. In one aspect the one or more flavours are present in a total amount of no greater than 5 wt. % based on the aerosolisable formulation. In one aspect the one or more flavours are present in a total amount of no greater than 4 wt. % based on the aerosolisable formulation. In one aspect the one or more flavours are present in a total amount of no greater than 3 wt. % based on the aerosolisable formulation. In one aspect the one or more flavours are present in a total amount of no greater than 2 wt. % based on the aerosolisable formulation. In one aspect the one or more flavours are present in a total amount of no greater than 1 wt. % based on the aerosolisable formulation.

In one aspect the one or more flavours are present in a total amount of from 0.01 to 5 wt. % based on the aerosolisable formulation. In one aspect the one or more flavours are present in a total amount of from 0.01 to 4 wt. % based on the aerosolisable formulation. In one aspect the one or more flavours are present in a total amount of from 0.01 to 3 wt. % based on the aerosolisable formulation. In one aspect the one or more flavours are present in a total amount of from 0.01 to 2 wt. % based on the aerosolisable formulation. In one aspect the one or more flavours are present in a total amount of from 0.01 to 1 wt. % based on the aerosolisable formulation. In one aspect the one or more flavours are present in a total amount of from 0.01 to 0.5 wt. % based on the aerosolisable formulation.

In one aspect the one or more flavours are present in a total amount of no greater than 10 wt. % based on the aerosolised formulation. In one aspect the one or more flavours are present in a total amount of no greater than 7 wt. % based on the aerosolised formulation. In one aspect the one or more flavours are present in a total amount of no greater than 5 wt. % based on the aerosolised formulation. In one aspect the one or more flavours are present in a total amount of no greater than 4 wt. % based on the aerosolised formulation. In one aspect the one or more flavours are present in a total amount of no greater than 3 wt. % based on the aerosolised formulation. In one aspect the one or more flavours are present in a total amount of no greater than 2 wt. % based on the aerosolised formulation. In one aspect the one or more flavours are present in a total amount of no greater than 1 wt. % based on the aerosolised formulation.

In one aspect the one or more flavours are present in a total amount of from 0.01 to 5 wt. % based on the aerosolised formulation. In one aspect the one or more flavours are present in a total amount of from 0.01 to 4 wt. % based on the aerosolised formulation. In one aspect the one or more flavours are present in a total amount of from 0.01 to 3 wt. % based on the aerosolised formulation. In one aspect the one or more flavours are present in a total amount of from 0.01 to 2 wt. % based on the aerosolised formulation. In one aspect the one or more flavours are present in a total amount of from 0.01 to 1 wt. % based on the aerosolised formulation. In one aspect the one or more flavours are present in a total amount of from 0.01 to 0.5 wt. % based on the aerosolised formulation.

### Formulation

One or more cyclodextrins may or may not be present in any suitable amount in the aerosolisable formulation. In one aspect the one or more cyclodextrins are present in a total amount of no greater than 12 wt. % based on the aerosolisable formulation. In one aspect the one or more cyclodextrins are present in a total amount of no greater than 10 wt. % based on the aerosolisable formulation. In one aspect the one or more cyclodextrins are present in a total amount of no greater than 9 wt. % based on the aerosolisable formulation. In one aspect the one or more cyclodextrins are present in a total amount of no greater than 8 wt. % based on the aerosolisable formulation. In one aspect the one or more cyclodextrins are present in a total amount of no greater than 7 wt. % based on the aerosolisable formulation. In one aspect the one or more cyclodextrins are present in a total amount of no greater than 6 wt. % based on the aerosolisable formulation. In one aspect the one or more cyclodextrins are present in a total amount of no greater than 5 wt. % based on the aerosolisable formulation. In one aspect the one or more cyclodextrins are present in a total amount of no greater than 4 wt. % based on the aerosolisable formulation. In one aspect the one or more cyclodextrins are present in a total amount of no greater than 3 wt. % based on the aerosolisable formulation. In one aspect the one or more cyclodextrins are present in a total amount of no greater than 2 wt. % based on the aerosolisable formulation. In one aspect the one or more cyclodextrins are present in a total amount of no greater than 1 wt. % based on the aerosolisable formulation. In one aspect the one or more cyclodextrins are present in a total amount of no greater than 0.5 wt. % based on the aerosolisable formulation. In one aspect the one or more cyclodextrins are present in a total amount of no greater than 0.1 wt. % based on the aerosolisable formulation. In one aspect the one or more cyclodextrins are present in a total amount of no greater than 0.01 wt. % based on the aerosolisable formulation. In one aspect the one or more cyclodextrins are present in a total amount of no greater than 0.001 wt. % based on the aerosolisable formulation.

One or more cyclodextrins may or may not be present in any suitable amount in the aerosolised formulation. In one aspect the one or more cyclodextrins are present in a total amount of no greater than 12 wt. % based on the aerosolised formulation. In one aspect the one or more cyclodextrins are present in a total amount of no greater than 10 wt. % based on the aerosolised formulation. In one aspect the one or more cyclodextrins are present in a total amount of no greater than 9 wt. % based on the aerosolised formulation. In one aspect the one or more cyclodextrins are present in a total amount of no greater than 8 wt. % based on the aerosolised formulation. In one aspect the one or more cyclodextrins are present in a total amount of no greater than 7 wt. % based on the aerosolised formulation. In one aspect the one or more cyclodextrins are present in a total amount of no greater than 6 wt. % based on the aerosolised formulation. In one aspect the one or more cyclodextrins are present in a total amount of no greater than 5 wt. % based on the aerosolised formulation. In one aspect the one or more cyclodextrins are present in a total amount of no greater than 4 wt. % based on the aerosolised formulation. In one aspect the one or more cyclodextrins are present in a total amount of no greater than 3 wt. % based on the aerosolised formulation. In one aspect the one or more cyclodextrins are present in a total amount of no greater than 2 wt. % based on the aerosolised formulation. In one aspect the one or more cyclodextrins are present in a total amount of no greater than 1 wt. % based on the aerosolised formulation. In one aspect the one or more cyclodextrins are present in a total amount of no greater than 0.5 wt. % based on the aerosolised formulation. In one aspect the one or more cyclodextrins are present in a total amount of no greater than 0.1 wt. % based on the aerosolised formulation. In one aspect the one or more cyclodextrins are present in a total amount of no greater than 0.01 wt. % based on the aerosolised formulation. In one aspect the one or more cyclodextrins are present in a total amount of no greater than 0.001 wt. % based on the aerosolised formulation.

The one or more cyclodextrins may be selected from the group consisting of unsubstituted cyclodextrins, substituted cyclodextrins and mixtures thereof. In one aspect at least one cyclodextrin is an unsubstituted cyclodextrin. In one aspect the one or more cyclodextrins are selected from the group consisting of unsubstituted cyclodextrins. In one aspect at least one cyclodextrin is a substituted cyclodextrin. In one aspect the one or more cyclodextrins are selected from the group consisting of substituted cyclodextrins.

In one aspect the one or more cyclodextrins are selected from the group consisting of unsubstituted (α)-cyclodextrin, substituted (α)-cyclodextrin, unsubstituted (β)-cyclodextrin, substituted (β)-cyclodextrin, unsubstituted (γ)-cyclodextrin, substituted (γ)-cyclodextrin, and mixtures thereof. In one aspect the one or more cyclodextrins are selected from the group consisting of unsubstituted (β)-cyclodextrin, substituted (β)-cyclodextrin, and mixtures thereof.

In one aspect the one or more cyclodextrins are selected from the group consisting of unsubstituted (α)-cyclodextrin, unsubstituted (β)-cyclodextrin, unsubstituted (γ)-cyclodextrin, and mixtures thereof. In one aspect the one or more cyclodextrins is selected from unsubstituted (β)-cyclodextrin.

In one aspect the one or more cyclodextrins are selected from the group consisting of substituted (α)-cyclodextrin, substituted (3)-cyclodextrin, substituted (γ)-cyclodextrin, and mixtures thereof. In one aspect the one or more cyclodextrins is selected from substituted (β)-cyclodextrins. Chemical substitutions at the 2-, 3—, and 6-hydroxyl sites are envisaged, and in particular substitution at the 2-position.

In one aspect the one or more cyclodextrins are selected from the group consisting of 2-hydroxy-propyl-α-cyclodextrin, 2-hydroxy-propyl-β-cyclodextrin, 2-hydroxy-propyl-γ-cyclodextrin and mixtures thereof. In one aspect the one or more cyclodextrins is at least 2-hydroxy-propyl-α-cyclodextrin. In one aspect the one or more cyclodextrins is at least 2-hydroxy-propyl-β-cyclodextrin. In one aspect the one or more cyclodextrins is at least 2-hydroxy-propyl-γ-cyclodextrin.

2-hydroxy-propyl derivatives of cyclodextrins, such as 2-hydroxy-propyl-β-cyclodextrin have increased solubility in water when compared to base cyclodextrins such as β-cyclodextrin. In one aspect if the aerosolisable formulation contains one or more cyclodextrins, then the aerosolisable formulation contains no flavours that can be encapsulated by the one or more cyclodextrins.

### Process

As discussed herein, in one aspect there is provided a process for forming an aerosol comprising
(a) at least one active agent;
(b) a carrier selected from water, propylene glycol, glycerol and mixtures thereof; and
(c) a modulator selected from a TRPA1 agonist, a TRPM8 agonist, a TRPV1 agonist,
a TRPV3 agonist, a TRPA1 antagonist, a TRPA1 inhibitor and combinations thereof,
   the process comprising one of
   (A) aerosolising an aerosolisable formulation comprising
(a) at least one active agent;
(b) a carrier selected from water, propylene glycol, glycerol and mixtures thereof; and
(c) a modulator selected from a TRPA1 agonist, a TRPM8 agonist, a TRPV1 agonist, a TRPV3 agonist, a TRPA1 antagonist, a TRPA1 inhibitor and combinations thereof
   or
   (B) aerosolising an aerosolisable formulation comprising
(a) at least one active agent; and
(b) a carrier selected from water, propylene glycol, glycerol and mixtures thereof; aerosolising an aerosolisable formulation comprising
(c) a modulator selected from a TRPA1 agonist, a TRPM8 agonist, a TRPV1 agonist, a TRPV3 agonist, a TRPA1 antagonist, a TRPA1 inhibitor and combinations thereof.

As will be appreciated, the process forms an aerosol containing the three components of the present formulation, namely (a) at least one active agent; (b) a carrier selected from water, propylene glycol, glycerol and mixtures thereof; and (c) a modulator selected from a TRPA1 agonist, a TRPM8 agonist, a TRPV1 agonist, a TRPV3 agonist, a TRPA1 antagonist, a TRPA1 inhibitor and combinations thereof.

In the process all 3 components may be formulated together and then formed into an aerosol or, component (a) and (b) may be formulated together and formed into an aerosol which is then contacted with component (c).

Thus, in one aspect, there is provided a process for forming an aerosol comprising
(a) at least one active agent;
(b) a carrier selected from water, propylene glycol, glycerol and mixtures thereof; and
(c) a modulator selected from a TRPA1 agonist, a TRPM8 agonist, a TRPV1 agonist,
a TRPV3 agonist, a TRPA1 antagonist, a TRPA1 inhibitor and combinations thereof,
   the process comprising aerosolising an aerosolisable formulation comprising
(a) at least one active agent;
(b) a carrier selected from water, propylene glycol, glycerol and mixtures thereof; and
(c) a modulator selected from a TRPA1 agonist, a TRPM8 agonist, a TRPV1 agonist,
a TRPV3 agonist, a TRPA1 antagonist, a TRPA1 inhibitor and combinations thereof.

Thus, in one aspect, there is provided a process for forming an aerosol comprising
(a) at least one active agent;
(b) a carrier selected from water, propylene glycol, glycerol and mixtures thereof; and
(c) a modulator selected from a TRPA1 agonist, a TRPM8 agonist, a TRPV1 agonist,
a TRPV3 agonist, a TRPA1 antagonist, a TRPA1 inhibitor and combinations thereof, the process comprising aerosolising an aerosolisable formulation comprising
(a) at least one active agent; and
(b) a carrier selected from water, propylene glycol, glycerol and mixtures thereof; aerosolising an aerosolisable formulation comprising
(c) a modulator selected from a TRPA1 agonist, a TRPM8 agonist, a TRPV1 agonist,
a TRPV3 agonist, a TRPA1 antagonist, a TRPA1 inhibitor and combinations thereof.

In one aspect of the process the aerosol may be formed by heating the formulation. For example, the aerosolisable formulation may be provided in a container, such as a bottle, or may be a component for use with an aerosol provision device. For example, the article may comprise an area (store) for receiving the aerosolisable formulation defined herein, an aerosol generating component, an aerosol generating area, and/or a mouthpiece. In some embodiments, there is provided an article for use with an aerosol provision system, the article comprising a store comprising an aerosolisable formulation as defined herein, an aerosol generating component (such as a heater), an aerosol generating area, a transport element, and a mouthpiece.

Aerosolisable formulation may be transferred from the store for receiving an aerosolisable formulation to the aerosol generating component via a transport element, such as a wick, pump or the like. The skilled person is able to select suitable transport elements depending on the type of aerosolisable formulation that is to be transported and the rate at which it must be supplied. Particular mention may be made of transport elements, such as wicks, formed from fibrous materials, foamed materials, sintered materials, woven and non-woven materials.

An airflow pathway typically extends through the article (optionally via the device) to an outlet. The pathway is oriented such that generated aerosol is entrained in the airflow such that it can be delivered to the outlet for inhalation by a user.

In one embodiment, the aerosol generating component is a heater.

In one embodiment, the aerosol generating component is a not a heater. In aspect of the process the aerosol may be formed by a process performed at a temperature below 60° C. In the process the aerosol may be formed by a process performed at a temperature below 50° C.

In the process the aerosol may be formed by a process performed at a temperature below 40° C. In the process the aerosol may be formed by a process performed at a temperature below 30° C. In the process the aerosol may be formed by a process performed at a temperature below 25° C. In the process the aerosol may be formed by a process which does not involve heating.

In the process the aerosol may be formed by applying ultrasonic energy to the aerosolisable formulation. In one embodiment, the aerosol generating component is an ultrasonic aerosol generator.

The formulation may be contained or delivered by any means. In one aspect the present invention provides a contained aerosolisable formulation comprising (a) one or more containers; and (b) an aerosolisable formulation as defined herein. The container may be any suitable container, for example to allow for the storage or delivery of the formulation. In one aspect the container is configured for engagement with an electronic aerosol provision system. The container may be configured to become fluidly in communication with an electronic aerosol provision system so that formulation may be delivered to the electronic aerosol provision system. As described above, the present disclosure relates to container which may be used in an electronic aerosol provision system, such as an e-cigarette. Throughout the following description the term “e-cigarette” is used; however, this term may be used interchangeably with electronic aerosol provision system.

The components may be provided together in a single formulation or may be provided separately for combination before inhalation. In one aspect there is provided a kit comprising (1) a first component comprising (i) a first container; and (ii) an aerosolisable formulation, comprising (a) at least one active agent; and (b) a carrier selected from water, propylene glycol, glycerol and mixtures thereof;
(2) a second component comprising (i) a second container; and (ii) a modulator formulation comprising a modulator selected from a TRPA1 agonist, a TRPM8 agonist, a TRPV1 agonist, a TRPV3 agonist, a TRPA1 antagonist, a TRPA1 inhibitor and combinations thereof.

As discussed herein, the container of the present invention is typically provided for the delivery of aerosolisable formulation to or within an e-cigarette. The aerosolisable formulation may be held within an e-cigarette or may be sold as a separate container for subsequent use with or in an e-cigarette. As understood by one skilled in the art, e-cigarettes may contain a unit known as a detachable cartomiser which typically comprises a reservoir of aerosolisable formulation, and an aerosoliser or aerosol-generator such as a wick material and a heating element for vaporising the aerosolisable formulation. In some e-cigarettes, the cartomiser is part of a single-piece device and is not detachable. In one aspect the container is a cartomiser or is part of a cartomiser. In one aspect the container is not a cartomiser or part of a cartomiser and is a container, such as a tank, which may be used to deliver nicotine formulation to or within an e-cigarette.

In one aspect the container is part of an e-cigarette. Therefore, in a further aspect the present invention provides an electronic aerosol provision system comprising: an aerosolisable formulation as defined herein; an aerosoliser or aerosol-generator for aerosolising formulation for inhalation by a user of the electronic aerosol provision system; and a power supply comprising a cell or battery for supplying power to the aerosoliser or aerosol-generator. There is provided an electronic aerosol provision system comprising: (i) an aerosoliser or aerosol-generator for aerosolising formulation for inhalation by a user of the electronic aerosol provision system; (ii) a power supply comprising a cell or battery for supplying power to the aerosoliser or aerosol-generator (iii) an aerosolisable formulation, comprising at least
(a) at least one active agent; and
(b) a carrier selected from water, propylene glycol, glycerol and mixtures thereof; (iv) a modulator selected from a TRPA1 agonist, a TRPM8 agonist, a TRPV1 agonist, a TRPV3 agonist, a TRPA1 antagonist, a TRPA1 inhibitor and combinations thereof. An aerosol generator is an apparatus configured to cause aerosol to be generated from the aerosolisable formulation. In some embodiments, the aerosol generator is a heater configured to subject the aerosolisable formulation to heat energy, so as to release one or more volatiles from the aerosolisable formulation to form an aerosol. In some embodiments, the aerosol generator is configured to cause an aerosol to be generated from the aerosolisable formulation without heating. For example, the aerosol generator may be configured to subject the aerosolisable formulation to one or more of vibration, increased pressure, or electrostatic energy. In one aspect, the modulator is present in the aerosolisable formulation. In one aspect, the modulator is separate to the aerosolisable formulation. In one aspect, the electronic aerosol provision system further comprises an acid. The acid may be present in the aerosolisable formulation or may be separate to the aerosolisable formulation.

In addition to the aerosolisable formulation of the present invention and to systems such as containers and electronic aerosol provision systems containing the same, the present invention provides use of a modulator for reducing the harshness when inhaled of an aerosolised formulation,
wherein the modulator is selected from a TRPA1 antagonist, a TRPM8 agonist, a TRPV1 agonist, a TRPV3 agonist, a TRPA1 antagonist, a TRPA1 inhibitor and combinations thereof; and
wherein the aerolised formulation comprises
(a) at least one active agent; and
(b) a carrier selected from water, propylene glycol, glycerol and mixtures thereof.

The process of the present invention may comprise additional steps either before the steps listed, after the steps listed or between one or more of the steps listed.

The invention will now be described with reference to the following non-limiting example.

### EXAMPLES

### Example 1

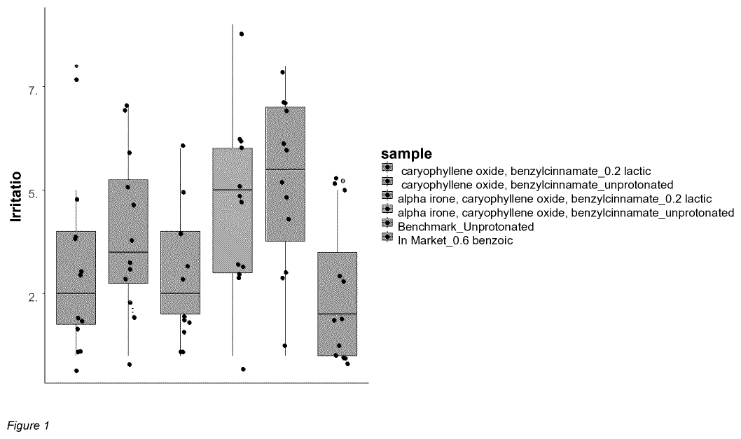

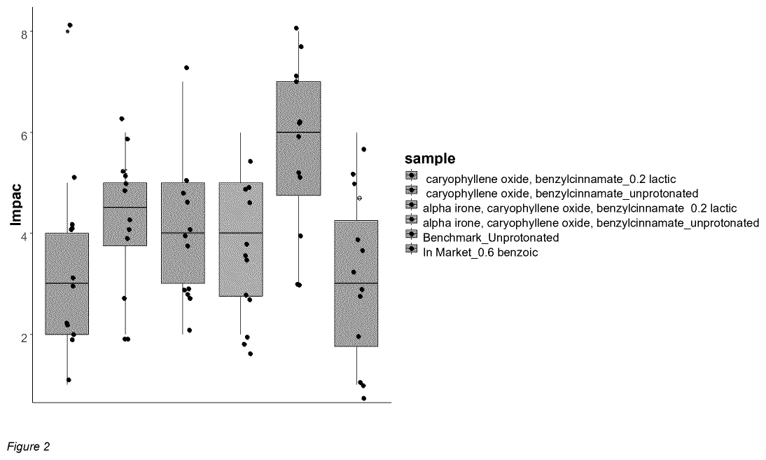

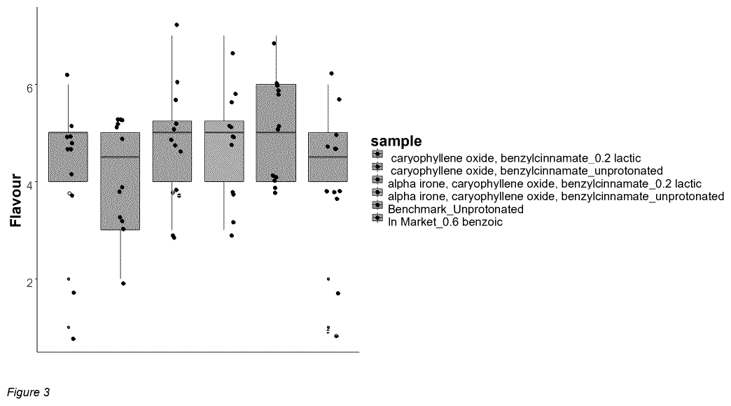

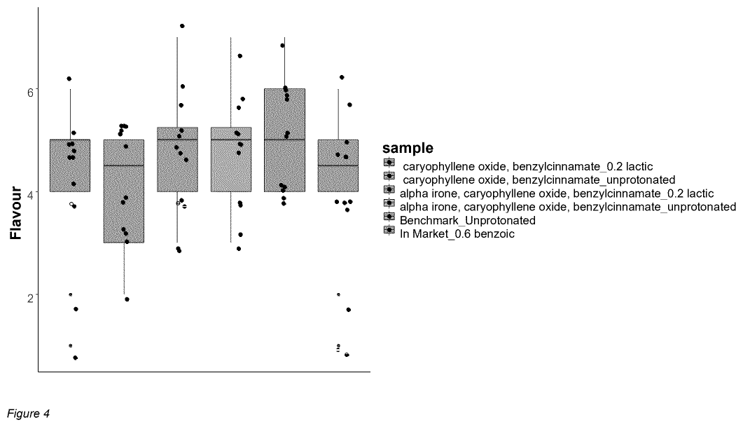

A product developer test (PDT) for e-liquids (a sensory test) with a mix of up to three TRP-receptor modulators (alpha-irone, caryophyllene oxide and benzyl cinnamate) was conducted using a sequential monadic scale (1-9) to evaluate the sensory performance of TRP-receptor modulators against unprotonated and protonated products. In doing so, 12 participants were asked to evaluate irritation, impact, flavour intensity and overall likeability of the provided products. The results for each of these assessments is given in FIGS. 1 to 4.

From the data we have concluded that products containing TRP-receptor modulators show a reduction in irritation. This was particularly pronounced for the TRP-receptor modulators vs the unprotonated product and moreover, increased flavour intensity and impact vs current protonated products (current in market, 0.6 benzoic).

### Example 2

### Test Design

A quantitative home placement consumer assessment was completed with 108 independent participants balance for age and gender. Participants used the product at home and completed questionnaires to evaluate each product. This study was managed by an independent third party consumer research agency and conducted to industry standards.

### Formulations

The prototypes were prepared to evaluate the effect of materials that were identified as active on the harshness receptors. The actives were caryophyllene oxide and benzyl cinnamate.
The same device was used for both samples.
The same instructions were provided.
There was a benchmark and a test sample.

The only differences were related to the presence of the two actives within the liquid as shown in Table 1.
The Benchmark product was an e-liquid formula containing propylene glycol, glycerine, nicotine and flavour.
The Test samples included the same 4 ingredients and in addition included benzyl cinnamate and caryophyllene oxide.

TABLE 1
Formulations for Benchmark and Test samples.

| | TOBACCO FLAVOUR | | MANGO FLAVOUR | |
|---|---|---|---|---|
| | Benchmark | Test | Benchmark | Test |
| | % w/w | % w/w | % w/w | % w/w |
| Propylene Glycol | 45.000 | 44.944 | 45.000 | 44.944 |
| Glycerine | 48.410 | 47.914 | 48.410 | 47.914 |
| Nicotine | 1.590 | 1.590 | 1.59 | 1.59 |
| Tobacco Flavour | 5.000 | 5 | 0 | 0 |
| Mango Flavour | 0.000 | 0 | 5 | 5 |
| Benzyl Cinnamate | 0.000 | 0.496 | 0.000 | 0.496 |
| Caryophyllene Oxide | 0.000 | 0.056 | 0.000 | 0.056 |
| Total | 100.000 | 100.000 | 100.000 | 100.000 |

### Consumer Evaluation

The consumers we asked to trial each product for a period of 1 day then complete an online quantitative questionnaire where they rated the product. Two difference scales were used:
1. Magnitude scale: 7 point scale used to rate sensation levels from low (1) to high (7).
2. Just Right scale: Evaluates how close to “Just Right” the sensation is. This is a 5 point scale
   Magnitude scale and Results

The magnitude scale poses a question and then provides a 1-7 point scale. The scale requires that that a ranking be provided based on the following question:
“Thinking about the e-liquid you vaped today how would you rate it for each of the following characteristics in terms of being low or high?”
Harshness in throat is then ranked from 1 [Low] to 7 [High]

The results are provided in Tables 2 and 3

TABLE 2
Magnitude ratings for Tobacco Benchmark and Prototypes

| | TOBACCO FLAVOUR | | |
|---|---|---|---|
| | Benchmark | Test | CONCLUSION |
| Average Rating for Level | 3.61 | 3.43 | Test product is rated |
| of irritation during | | | lower for irritation |
| vaping session | | | vs theBenchmark |

TABLE 3
Magnitude ratings for Mango Benchmark and Prototypes

| | MANGO FLAVOUR | | |
|---|---|---|---|
| | Benchmark | Test | CONCLUSION |
| Average Rating for Level | 3.78 | 3.49 | Test product is rated |
| of irritation during | | | lower for irritation |
| vaping session | | | vs the Benchmark |

### Just Right Scale and Results

The rating scale used is shown below and measured how “just right” the sample is for the “Level of Irritation during Vaping Session”. The possible ratings were:
−2 Much less than I wanted
−1 Slightly less than I wanted
0 Just Right
+1 Slightly more than I wanted
+2 Much more than I wanted

TABLE 4
Just right ratings for Tobacco Benchmark and Prototypes

| | TOBACCO FLAVOUR | | |
|---|---|---|---|
| | Benchmark | Test | Conclusion |
| Average Rating for level | 0.44 | 0.39 | Test product is |
| of Irritation during | | | closer to zero than |
| vaping session | | | the benchmark |
| Average Rating for | −0.44 | −0.22 | Test product is |
| smoothness of Vapour | | | closer to zero than |
| | | | the benchmark |

TABLE 5
Just Right ratings for Mango Benchmark and Prototypes

| | MANGO FLAVOUR | | |
|---|---|---|---|
| | Benchmark | Test | Conclusion |
| Average Rating for level | 0.49 | 0.27 | Test product is |
| of Irritation during | | | closer to zero than |
| vaping session | | | the benchmark |
| Average Rating for | −0.36 | −0.19 | Test product is |
| smoothness of Vapour | | | closer to zero than |
| | | | the benchmark |

### Overall Conclusion

The presence of benzyl cinnamate and caryophyllene oxide results in products that are less irritating and more “Just Right” for level of irritation and level of smoothness of the vapour.

The invention will be described in further detail in the following numbered embodiments.

1. An aerosolisable formulation or aerosol formulation comprising
(a) at least one active agent;
(b) a carrier selected from water, propylene glycol, glycerol and mixtures thereof; and
(c) a modulator selected from a TRPA1 agonist, a TRPM8 agonist, a TRPV1 agonist, a TRPV3 agonist, a TRPA1 antagonist, a TRPA1 inhibitor and combinations thereof; OR an aerosolisable formulation or aerosol formulation comprising
(a) at least one active agent;
(b) aerosol former material; and
(c) a modulator selected from a TRPA1 agonist, a TRPM8 agonist, a TRPV1 agonist, a TRPV3 agonist, a TRPA1 antagonist, a TRPA1 inhibitor and combinations thereof.

2. An aerosolisable formulation or aerosol formulation according to embodiment 1 wherein the modulator comprises at least one or more of a TRPA1 agonist, a TRPM8 agonist, a TRPV1 agonist, and a TRPV3 agonist.

3. An aerosolisable formulation or aerosol formulation according to embodiment 1 or 2 wherein the modulator comprises at least a TRPA1 antagonist or a TRPA1 inhibitor.

4. An aerosolisable formulation or aerosol formulation according to embodiment 1, 2 or 3 wherein the modulator comprises at least
(i) one or more of a TRPM8 agonist, a TRPV1 agonist, and a TRPV3 agonist; and
(ii) an TRPA1 antagonist or a TRPA1 inhibitor.

5. An aerosolisable formulation or aerosol formulation according to any one of embodiments 1 to 4 wherein the TRPA1 antagonist or a TRPA1 inhibitor has a stronger binding affinity to the TRPA1 receptor than the binding affinity of the one or more of a TRPM8 agonist, a TRPV1 agonist, and a TRPV3 agonist to their respective receptor.

6. An aerosolisable formulation or aerosol formulation according to any one of embodiments 1 to 5 wherein the modulator comprises at least
(i) a TRPM8 agonist or a TRPV1 agonist; and
(ii) a TRPA1 antagonist or a TRPA1 inhibitor.

7. An aerosolisable formulation or aerosol formulation according to any one of embodiments 1 to 6 wherein the modulator comprises at least
(i) a TRPM8 agonist and a TRPV1 agonist; and
(ii) a TRPA1 antagonist or a TRPA1 inhibitor.

8. An aerosolisable formulation or aerosol formulation according to any one of embodiments 1 to 7 wherein the modulator is a single compound.

9. An aerosolisable formulation or aerosol formulation according to any one of embodiments 1 to 8 wherein the TRPA1 antagonist binds with one or more sites selected from Arg852, Gln979, His983, Ile858, Leu982, Met978, Trp711, Val861, Val967, Ala836, Gln940, Ile837, Leu847, Leu848, Leu863, Leu867, Leu871, Met844, Phe841, Phe884, Phe947, Ser887, Tyr840; and combinations thereof.

10. An aerosolisable formulation or aerosol formulation according to any one of embodiments 1 to 9 wherein the TRPA1 antagonist binds with one or more sites selected from Arg852, Gln979, His983, Ile858, Leu982, Met978, Trp711, Val861, Val967; and combinations thereof.

11. An aerosolisable formulation or aerosol formulation according to any one of embodiments 1 to 10 wherein the TRPA1 agonist binds with one or more sites selected from Ala836, Gln940, Ile837, Leu847, Leu848, Leu863, Leu867, Leu871, Met844, Phe841, Phe884, Phe947, Ser887, Tyr840; and combinations thereof.

12. An aerosolisable formulation or aerosol formulation according to any one of embodiments 1 to 11 wherein the TRPM8 agonist binds with one or more sites selected from Arg1007, Arg841, Asn741, Asp781, Ile845, Leu1000, Phe738, Tyr1004, Tyr745, Val848; and combinations thereof.

13. An aerosolisable formulation or aerosol formulation according to any one of embodiments 1 to 12 wherein the TRPV1 agonist binds with one or more sites selected from Ala548, Ala568, Ala667, Asn553, Glu572, Ile571, Ile663, Leu517, Leu555, Leu664, Leu671, Met549, Phe518, Phe545, Phe593, Ser514, Thr552, Tyr513, Tyr556; and combinations thereof.

14. An aerosolisable formulation or aerosol formulation according to any one of embodiments 1 to 13 wherein the TRPV3 agonist binds with one or more sites selected from Arg693, His430, His426, His417, Leu420, Leu694, Leu429, Trp433, Thr421; and combinations thereof.

15. An aerosolisable formulation or aerosol formulation according to any one of embodiments 1 to 14 wherein the TRPA1 inhibitor binds with one or more sites selected from Leu867, Leu870, Leu871, Ile946, Ser873 Thr873, Thr874, Phe944, Val948, Phe877, Ile878, Leu880, Leu881, Met912, Phe909, Thr908, Ile906, Ile905, Ile950, Leu956, Val942, Met953, Leu952, Phe884; and combinations thereof.

16. An aerosolisable formulation or aerosol formulation according to any one of embodiments 1 to 15 wherein the TRPA1 antagonist is selected from cinnamic acid, cinnamyl ester; hexylresorcinol; I-menthol; 2-ethyl-6-methoxyphenol; linalyl oxide; 2-methoxy-6-methylphenol; methyl 2-(methylamino)benzoate; eucalyptol; and mixtures thereof.

17. An aerosolisable formulation or aerosol formulation according to any one of embodiments 1 to 16 wherein the TRPM8 agonist is selected from (1Z,4E,8E)-2,6,6,9-tetramethylcycloundeca-1,4,8-triene; beta-caryophyllene; liquiritin; ambrox; (E)-2-epi-beta-caryophyllene; bicyclo[7.2.0]undec-4-ene, 4,11,11-trimethyl-8-methylene-, (1R,4E,9S)—; (4Z)-4, 11,11-trimethyl-8-methylidenebicyclo[7.2.0]undec-4-ene; bicyclo[7.2.0]undec-4-ene, 4,11,11-trimethyl-8-methylene-, (1S,4E,9R)—; isocaryophyllene; ambroxan; spatulenol; caryophyllene oxide; (−)-beta-bourbonene; oxacycloheptadec-10-en-2-one, (E)-; cubebol; trans-caryophyllene; gamma-dodecalactone; hydroxy-alpha-sanshool; citronellyl acetate; (2E,6Z,8E,10E)-N-(2-Hydroxy-2-methylpropyl) dodeca-2,6,8,10-tetraenamide; (8E,10E)-N-(2-hydroxy-2-methylpropyl) dodeca-2,6,8, 10-tetraenamide; N-(2-Hydroxy-2-methylpropyl) dodeca-2,6,8, 10-tetraenamide; hydroxy-beta-sanshool; 2-hexenal; and mixtures thereof.

18. An aerosolisable formulation or aerosol formulation according to any one of embodiments 1 to 17 wherein the TRPV1 agonist is selected from phenethyl phenylacetate; 2-propenoic acid, 3-phenyl-, 3-phenyl-2-propenyl ester; theaspirane; (7R,11R)-3,7,11,15-tetramethylhexadec-2-en-1-ol; 4-(2,5,6,6-tetramethylcyclohex-2-en-1-yl)but-3-en-2-one; cinnamyl cinnamate; alpha-irone; beta-spathulenol; spathulenol; phytol; 1-penten-3-one, 1-[(1R)-2,6,6-trimethyl-2-cyclohexen-1-yl]-, (1E)-; (S)-alpha-Methylionone; Guaiol; 3,7,11,15-tetramethylhexadec-2-en-1-ol; 3,7, 11,15-tetramethyl-2-hexadecen-1-ol; (Z)-3,7,11,15-tetramethylhexadec-2-en-1-ol; vanillyl butyl ether; 4-ethyl-2-methoxyphenol; 6-methyl-2-(oxiran-2-yl)hept-5-en-2-ol; 2-methoxy-4-methylphenol; 2-methoxy-4-vinylphenol; benzyl cinnamate; 2-propenoic acid; 3-phenyl-, phenylmethyl ester; and mixtures thereof.

19. An aerosolisable formulation or aerosol formulation according to any one of embodiments 1 to 18 wherein the TRPV3 agonist is selected from ethyl vanillin; 2,6-dimethoxyphenol; vanillin; and mixtures thereof.

20. An aerosolisable formulation or aerosol formulation according to any one of embodiments 1 to 19 wherein the TRPA1 inhibitor is selected from 3-phenylpropyl homovanillate; benzyl alcohol, cinnamate; benzyl phenylacetate; alpha-amylcinnamaldehyde; linalyl benzoate; phenethyl isovalerate; carvacrol; thymol; 5-ethyl-2-methoxyphenol; vanitrope; and mixtures thereof.

21. An aerosolisable formulation or aerosol formulation according to any one of embodiments 1 to 20 wherein the TRPA1 antagonist is selected from cinnamyl cinnamate, liquiritin, 3-phenylpropyl homovanillate, benzyl cinnamate, caryophyllene (beta-), phenethyl phenylacetate, hydroxy-alpha-sanshool, phytol, bourbonene (beta) and mixtures thereof.

22. An aerosolisable formulation or aerosol formulation according to any one of embodiments 1 to 21 wherein the TRPA1 inhibitor is selected from cinnamyl cinnamate, citronellyl acetate, eucalyptol, 2-hexenal, dodecalactone (gamma-), menthol (I-), spathulenol, bourbonene (beta), benzyl cinnamate, caryophyllene oxide, caryophyllene (beta-), alpha-irone, phenethyl phenylacetate, methyl-alpha-ionone, phytol and mixtures thereof.

23. An aerosolisable formulation or aerosol formulation according to any one of embodiments 1 to 21 wherein the modulator is at least caryophyllene oxide, benzyl cinnamate, or a mixture thereof.

24. An aerosolisable formulation or aerosol formulation according to any one of embodiments 1 to 21 wherein the modulator is at least a mixture of caryophyllene oxide and benzyl cinnamate.

25. An aerosolisable formulation or aerosol formulation according to any one of embodiments 1 to 24 wherein the active agent is at least nicotine.

26 An aerosolisable formulation or aerosol formulation according to any one of embodiments 1 to 25 wherein the active agent is present in an amount of no greater than 6 wt. % based on the aerosolisable formulation or aerosol formulation.

27. An aerosolisable formulation or aerosol formulation according to embodiment 26 wherein the active agent is present in an amount of from 0.01 to 5 wt. % based on the aerosolisable formulation or aerosol formulation.

28. An aerosolisable formulation or aerosol formulation according to any one of embodiments 1 to 27 further comprising at least one acid.

29. An aerosolisable formulation or aerosol formulation according to embodiment 28 wherein the acid is selected from the group consisting of acetic acid, lactic acid, formic acid, citric acid, benzoic acid, pyruvic acid, levulinic acid, succinic acid, tartaric acid, sorbic acid, propionic acid, phenylacetic acid, salicylic acid, malic acid, caffeic acid, fumaric acid, nicotinic acid, o-toluic acid, m-toluic acid, p-toluic acid, capric acid, glucono delta-lactone, gluconic acid, malonic acid, phosphoric acid, 4-hydroxyphenylacetic acid and mixtures thereof.

30. An aerosolisable formulation or aerosol formulation according to embodiment 28 wherein the acid is selected from the group consisting of salicylic acid, malic acid, citric acid, succinic acid, caffeic acid, fumaric acid, nicotinic acid, o-toluic acid, m-toluic acid, p-toluic acid, capric acid, glucono delta-lactone, pyruvic acid, sorbic acid, levulinic acid, tartaric acid, gluconic acid, malonic acid, phosphoric acid, 4-hydroxyphenylacetic acid and mixtures thereof.

31. An aerosolisable formulation or aerosol formulation according to embodiment 28, 29 or 30 wherein the total content of acid present in the formulation is no greater than 1.0 mole equivalents based on the nicotine.

32. An aerosolisable formulation or aerosol formulation according to any one of embodiments 28 to 31 wherein the total content of acid present in the solution is no less than 0.1 mole equivalents based on the nicotine.

33. An aerosolisable formulation according to any one of embodiments 1 to 32 in the form of a solid, semi-solid or gel.

34. An aerosolisable formulation according to embodiment 33 comprising one or more binders.

35 An aerosolisable formulation comprising
(a) at least one active agent;
(b) aerosol former material;
(c) a modulator selected from a TRPA1 agonist, a TRPM8 agonist, a TRPV1 agonist, a TRPV3 agonist, a TRPA1 antagonist, a TRPA1 inhibitor and combinations thereof; and
(d) one or more binders.

36. An aerosolisable formulation according to embodiment 33, 34 or 35 comprising the one or more binders in an amount of 20 to 40 wt % based on the dry weight basis of the aerosolisable formulation.

37. An aerosolisable formulation according to any one of embodiments 33 to 36 wherein the one or more binders comprise one or more compounds selected from polysaccharide gelling agents, such as alginate, pectin, starch or a derivative thereof, cellulose or a derivative thereof, pullulan, carrageenan, agar and agarose; gelatin; gums, such as xanthan gum, guar gum and acacia gum; silica or silicone compounds, such as PDMS and sodium silicate; clays, such as kaolin; and polyvinyl alcohol.

38. An aerosolisable formulation according to embodiment 37 wherein the polysaccharide gelling agent is selected from alginate and a cellulose derivative.

39. An aerosolisable formulation according to embodiment 37 or 38 wherein the cellulose derivative is selected from microcrystalline cellulose (MCC), hydroxymethyl cellulose, hydroxyethyl cellulose, hydroxypropyl cellulose, carboxymethylcellulose (CMC), hydroxypropyl methylcellulose (HPMC), methyl cellulose, ethyl cellulose, cellulose acetate (CA), cellulose acetate butyrate (CAB), and cellulose acetate propionate (CAP).

40. An aerosolisable formulation according to any one of embodiments 33 to 39 wherein the one or more binders is not crosslinked.

41. An aerosolisable formulation according to any one of embodiments 33 to 40 wherein the one or more binders is MCC and/or CMC.

42. An aerosolisable formulation or aerosol formulation according to any one of embodiments 1 to 27, or an aerosolisable formulation according to embodiment 35, wherein the aerosol former material comprises one or more of glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

43. An aerosolisable formulation according to embodiment 42 wherein the aerosol-former material comprises glycerol or a combination of glycerol and propylene glycol.

44. An aerosolisable formulation according to any one of embodiments 35 to 43 wherein the modulator is a modulator as defined in any one of embodiments 2 to 24.

45. An aerosol generating composition comprising an aerosolisable formulation according to any one of embodiments 1 to 44.

46. An aerosol generating composition according to embodiment 45 comprising from 50 to 100 wt % (WWB) of the aerosol generating material.

47. An aerosol generating composition comprising
(a) botanical material;
(b) an aerosol former; and
(c) a modulator selected from selected from a TRPA1 agonist, a TRPM8 agonist, a TRPV1 agonist, a TRPV3 agonist, a TRPA1 antagonist, a TRPA1 inhibitor and combinations thereof.

48. An aerosol generating composition according to embodiment 47 wherein the botanical material is a material from a plant in the tobacco family.

49. An aerosol generating composition according to embodiment 47 or 48 wherein the aerosol-former material comprises one or more of glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

50. An aerosol generating composition according to embodiment 49 wherein the aerosol-former material comprises glycerol or a combination of glycerol and propylene glycol.

51. An aerosol generating composition according to embodiment 47 wherein the aerosol former is selected from water, propylene glycol, glycerol and mixtures thereof.

52. An aerosol generating according to any one of embodiments 47 to 52 wherein the modulator is a modulator as defined in any one of embodiments 2 to 24.

53. A consumable for use in a non-combustible aerosol provision device, the consumable comprising an aerosolisable formulation or aerosol generating composition as defined in any one of embodiments 1 to 52.

54. A non-combustible aerosol provision system comprising the consumable of embodiment 53 and a non-combustible aerosol provision device, the non-combustible aerosol provision device comprising an aerosol-generation device arranged to generate aerosol from the consumable when the consumable is used with the non-combustible aerosol provision device.

55. Use of an aerosolisable formulation or aerosol generating composition as defined in any one of embodiments 1 to 52 in a consumable for use with a non-combustible aerosol provision device, the non-combustible aerosol provision device comprising an aerosol-generation device arranged to generate aerosol from the consumable when the consumable is used with the non-combustible aerosol provision device.

56. A process for forming an aerosol comprising
(a) at least one active agent;
(b) a carrier selected from water, propylene glycol, glycerol and mixtures thereof; and
(c) a modulator selected from a TRPA1 agonist, a TRPM8 agonist, a TRPV1 agonist,
a TRPV3 agonist, a TRPA1 antagonist, a TRPA1 inhibitor and combinations thereof, the process comprising one of
   (A) aerosolising an aerosolisable formulation comprising
(a) at least one active agent;
(b) a carrier selected from water, propylene glycol, glycerol and mixtures thereof; and
(c) a modulator selected from a TRPA1 agonist, a TRPM8 agonist, a TRPV1 agonist, a TRPV3 agonist, a TRPA1 antagonist, a TRPA1 inhibitor and combinations thereof
   or
   (B) aerosolising an aerosolisable formulation comprising
(a) at least one active agent; and
(b) a carrier selected from water, propylene glycol, glycerol and mixtures thereof; aerosolising an aerosolisable formulation comprising
(c) a modulator selected from a TRPA1 agonist, a TRPM8 agonist, a TRPV1 agonist,
a TRPV3 agonist, a TRPA1 antagonist, a TRPA1 inhibitor and combinations thereof.

57. A process according to embodiment 56 comprising

(B) aerosolising an aerosolisable formulation comprising
(a) at least one active agent; and
(b) a carrier selected from water, propylene glycol, glycerol and mixtures thereof; to form an initial aerosol and contacting the initial aerosol with
(c) a modulator selected from a TRPA1 agonist, a TRPM8 agonist, a TRPV1 agonist, a TRPV3 agonist, a TRPA1 antagonist, a TRPA1 inhibitor and combinations thereof.

58. A process according to embodiment 56 or 57 characterised by the features of any one of embodiments 2 to 32.

59. A contained aerosolisable formulation comprising
(i) a container; and
(ii) an aerosolisable formulation, comprising
   (a) at least one active agent;
   (b) a carrier selected from water, propylene glycol, glycerol and mixtures thereof; and
   (c) a modulator selected from a TRPA1 agonist, a TRPM8 agonist, a TRPV1 agonist,
   a TRPV3 agonist, a TRPA1 antagonist, a TRPA1 inhibitor and combinations thereof.

60. A contained aerosolisable formulation according to embodiment 59 wherein the aerosolisable formulation is an aerosolisable formulation as defined in any one of embodiments 2 to 32.

61. A contained aerosolisable formulation according to embodiment 59 or 60 wherein the container is configured for engagement with an aerosol provision system.

62. A contained aerosolisable formulation according to embodiment 59, 60 or 61 wherein the container is configured for engagement with an electronic aerosol provision system.

63. A kit comprising
(1) a first component comprising
(i) a first container; and
(ii) an aerosolisable formulation, comprising
   (a) at least one active agent; and
   (b) a carrier selected from water, propylene glycol, glycerol and mixtures thereof;
(2) a second component comprising
   (i) a second container; and
   (ii) a modulator formulation comprising a modulator selected from a TRPA1 agonist, a TRPM8 agonist, a TRPV1 agonist, a TRPV3 agonist, a TRPA1 antagonist, a TRPA1 inhibitor and combinations thereof.

64. An electronic aerosol provision system comprising:
(i) an aerosoliser for aerosolising formulation for inhalation by a user of the electronic aerosol provision system;
(ii) a power supply comprising a cell or battery for supplying power to the aerosoliser (iii) an aerosolisable formulation, comprising at least
   (a) at least one active agent; and
   (b) a carrier selected from water, propylene glycol, glycerol and mixtures thereof;
(iv) a modulator selected from a TRPA1 agonist, a TRPM8 agonist, a TRPV1 agonist, a TRPV3 agonist, a TRPA1 antagonist, a TRPA1 inhibitor and combinations thereof.

65. An electronic aerosol provision system according to embodiment 64 wherein the modulator is present in the aerosolisable formulation.

66. An electronic aerosol provision system according to embodiment 64 or 65 wherein the electronic aerosol provision system further comprises an acid.

67. An electronic aerosol provision system according to embodiment 66 wherein the acid is present in the aerosolisable formulation.

68. An electronic aerosol provision system according to any one of embodiments 64 to 67 wherein the aerosolisable formulation is an aerosolisable formulation as defined in any one of embodiments 2 to 32.

69. Use of a modulator for reducing the harshness when inhaled of an aerosolised formulation, wherein the modulator is selected from a TRPA1 agonist, a TRPM8 agonist, a TRPV1 agonist, a TRPV3 agonist, a TRPA1 antagonist, a TRPA1 inhibitor and combinations thereof; and
wherein the aerosolised formulation comprises
(a) at least one active agent; and
(b) a carrier selected from water, propylene glycol, glycerol and mixtures thereof.

70. Use according to embodiment 69 wherein the aerosolised formulation is formed from an aerosolisable formulation as defined in any one of embodiments 2 to 32.

71. An aerosolisable formulation comprising
(a) at least one active agent;
(b) aerosol former material;
(c) a modulator selected from a TRPA1 agonist, a TRPM8 agonist, a TRPV1 agonist, a TRPV3 agonist, a TRPA1 antagonist, a TRPA1 inhibitor and combinations thereof; and
(d) one or more binders.

72. An aerosol generating composition comprising
(a) botanical material;
(b) an aerosol former; and
(c) a modulator selected from selected from a TRPA1 agonist, a TRPM8 agonist, a TRPV1 agonist, a TRPV3 agonist, a TRPA1 antagonist, a TRPA1 inhibitor and combinations thereof.

73. An aerosol generating composition according to embodiment 72 wherein the botanical material is a material from a plant in the tobacco family.

74. An aerosol generating composition according to embodiment 72 or 73 further comprising one or more binders and optionally a filler.

75. An aerosol generating composition according to embodiment 71 or 74, wherein the one or more binders consists of one or more gelling agent(s).

76. An aerosolisable formulation or aerosol generating according to any one of embodiments 71 to 75 wherein the modulator comprises at least
(iii) one or more of a TRPM8 agonist, a TRPV1 agonist, and a TRPV3 agonist; and
(iv) an TRPA1 antagonist or a TRPA1 inhibitor.

77. An aerosolisable formulation or aerosol generating according to any one of embodiments 71 to 76 wherein the TRPA1 antagonist or a TRPA1 inhibitor has a stronger binding affinity to the TRPA1 receptor than the binding affinity of the one or more of a TRPM8 agonist, a TRPV1 agonist, and a TRPV3 agonist to their respective receptor.

78. An aerosolisable formulation or aerosol generating according to any one of embodiments 71 to 77 wherein the modulator comprises at least
(iii) a TRPM8 agonist or a TRPV1 agonist; and
(iv) a TRPA1 antagonist or a TRPA1 inhibitor.

79. An aerosolisable formulation or aerosol generating according to any one of embodiments 71 to 78 wherein the modulator comprises at least
(iii) a TRPM8 agonist and a TRPV1 agonist; and
(iv) a TRPA1 antagonist or a TRPA1 inhibitor.

80. An aerosolisable formulation or aerosol generating according to any one of embodiments 71 to 79 wherein the TRPA1 antagonist binds with one or more sites selected from Arg852, Gln979, His983, Ile858, Leu982, Met978, Trp711, Val861, Val967, Ala836, Gln940, Ile837, Leu847, Leu848, Leu863, Leu867, Leu871, Met844, Phe841, Phe884, Phe947, Ser887, Tyr840; and combinations thereof.

81. An aerosolisable formulation or aerosol generating according to any one of embodiments 71 to 80 wherein the TRPA1 antagonist binds with one or more sites selected from Arg852, Gln979, His983, Ile858, Leu982, Met978, Trp711, Val861, Val967; and combinations thereof.

82. An aerosolisable formulation or aerosol generating according to any one of embodiments 71 to 81 wherein the TRPA1 agonist binds with one or more sites selected from Ala836, Gln940, Ile837, Leu847, Leu848, Leu863, Leu867, Leu871, Met844, Phe841, Phe884, Phe947, Ser887, Tyr840; and combinations thereof.

83. An aerosolisable formulation or aerosol generating according to any one of embodiments 71 to 82 wherein the TRPM8 agonist binds with one or more sites selected from Arg1007, Arg841, Asn741, Asp781, Ile845, Leu1000, Phe738, Tyr1004, Tyr745, Val848; and combinations thereof.

84. An aerosolisable formulation or aerosol generating according to any one of embodiments 71 to 83 wherein the TRPV1 agonist binds with one or more sites selected from Ala548, Ala568, Ala667, Asn553, Glu572, Ile571, Ile663, Leu517, Leu555, Leu664, Leu671, Met549, Phe518, Phe545, Phe593, Ser514, Thr552, Tyr513, Tyr556; and combinations thereof.

85. An aerosolisable formulation or aerosol generating according to any one of embodiments 71 to 84 wherein the TRPV3 agonist binds with one or more sites selected from Arg693, His430, His426, His417, Leu420, Leu694, Leu429, Trp433, Thr421; and combinations thereof.

86. An aerosolisable formulation or aerosol generating according to any one of embodiments 71 to 85 wherein the TRPA1 inhibitor binds with one or more sites selected from Leu867, Leu870, Leu871, Ile946, Ser873 Thr873, Thr874, Phe944, Val948, Phe877, Ile878, Leu880, Leu881, Met912, Phe909, Thr908, Ile906, Ile905, Ile950, Leu956, Val942, Met953, Leu952, Phe884; and combinations thereof.

87. An aerosolisable formulation or aerosol generating according to any one of embodiments 71 to 86 wherein the active agent is at least nicotine and/or wherein the aerosolisable formulation or aerosol generating composition further comprising at least one acid.

88. An aerosolisable formulation or aerosol generating according to any one of embodiments 71 to 87 wherein the aerosol former material comprises one or more of glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate; such as wherein the aerosol-former material comprises glycerol or a combination of glycerol and propylene glycol.

89. A consumable for use in a non-combustible aerosol provision device, the consumable comprising an aerosolisable formulation or aerosol generating composition as defined in any one of embodiments 71 to 88.

90. A non-combustible aerosol provision system comprising the consumable of embodiment 89 and a non-combustible aerosol provision device, the non-combustible aerosol provision device comprising an aerosol-generation device arranged to generate aerosol from the consumable when the consumable is used with the non-combustible aerosol provision device.

91. Use of an aerosolisable formulation or aerosol generating composition as defined in any one of embodiments 71 to 88 in a consumable for use with a non-combustible aerosol provision device, the non-combustible aerosol provision device comprising an aerosol-generation device arranged to generate aerosol from the consumable when the consumable is used with the non-combustible aerosol provision device.

92. A process for forming an aerosol comprising
(a) at least one active agent;
(b) an aerosol former material; and
(c) a modulator selected from a TRPA1 agonist, a TRPM8 agonist, a TRPV1 agonist,
a TRPV3 agonist, a TRPA1 antagonist, a TRPA1 inhibitor and combinations thereof,
   the process comprising one of
   (A) aerosolising an aerosolisable formulation comprising
(a) at least one active agent;
(b) an aerosol former material; and
(c) a modulator selected from a TRPA1 agonist, a TRPM8 agonist, a TRPV1 agonist, a TRPV3 agonist, a TRPA1 antagonist, a TRPA1 inhibitor and combinations thereof
   or
   (B) aerosolising an aerosolisable formulation comprising
(a) at least one active agent; and
(b) a carrier selected from water, propylene glycol, glycerol and mixtures thereof; aerosolising an aerosolisable formulation comprising
(c) a modulator selected from a TRPA1 agonist, a TRPM8 agonist, a TRPV1 agonist,
a TRPV3 agonist, a TRPA1 antagonist, a TRPA1 inhibitor and combinations thereof;
   or
   (C) aerosolising an aerosolisable formulation comprising
(a) at least one active agent;
(b) aerosol former material;
(c) a modulator selected from a TRPA1 agonist, a TRPM8 agonist, a TRPV1 agonist,
a TRPV3 agonist, a TRPA1 antagonist, a TRPA1 inhibitor and combinations thereof; and
(d) one or more binders;
   or
   (D) aerosolising an aerosol generating composition comprising
(a) botanical material;
(b) an aerosol former; and
(c) a modulator selected from selected from a TRPA1 agonist, a TRPM8 agonist, a TRPV1 agonist, a TRPV3 agonist, a TRPA1 antagonist, a TRPA1 inhibitor and combinations thereof.

93. A kit comprising
(1) a first component comprising
(i) a first container; and
(ii) an aerosolisable formulation, comprising
   (a) at least one active agent; and
   (b) aerosol former material;
(2) a second component comprising
(i) a second container; and
(ii) a modulator formulation comprising a modulator selected from a TRPA1 agonist, a TRPM8 agonist, a TRPV1 agonist, a TRPV3 agonist, a TRPA1 antagonist, a TRPA1 inhibitor and combinations thereof.

94. An electronic aerosol provision system comprising:
(i) an aerosol-generator for aerosolising formulation for inhalation by a user of the electronic aerosol provision system;
(ii) a power supply comprising a cell or battery for supplying power to the aerosol-generator
(iii) an aerosolisable formulation, comprising at least
   (a) at least one active agent; and
   (b) aerosol former material;
(iv) a modulator selected from a TRPA1 agonist, a TRPM8 agonist, a TRPV1 agonist, a TRPV3 agonist, a TRPA1 antagonist, a TRPA1 inhibitor and combinations thereof.

95. Use of a modulator for reducing the harshness when inhaled of an aerosolised formulation,
wherein the modulator is selected from a TRPA1 agonist, a TRPM8 agonist, a TRPV1 agonist, a TRPV3 agonist, a TRPA1 antagonist, a TRPA1 inhibitor and combinations thereof; and
wherein the aerosolised formulation comprises
(a) at least one active agent; and
(b) aerosol former material.

Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or related fields are intended to be within the scope of the following claims.

1. An aerosolizable formulation comprising
(a) at least one active agent;
(b) aerosol former material;
(c) a modulator selected from a TRPA1 agonist, a TRPM8 agonist, a TRPV1 agonist, a TRPV3 agonist, a TRPA1 antagonist, a TRPA1 inhibitor and combinations thereof; and
(d) one or more binders.

2. The aerosolisable formulation according to claim 1 wherein the active agent is at least nicotine.

3. The aerosolisable formulation according to claim 1 wherein the active agent is at least a cannabinoid.

4. An aerosol generating composition comprising
(a) botanical material;
(b) an aerosol former; and
(c) a modulator selected from selected from a TRPA1 agonist, a TRPM8 agonist, a TRPV1 agonist, a TRPV3 agonist, a TRPA1 antagonist, a TRPA1 inhibitor and combinations thereof.

5. The aerosol generating composition according to claim 4 wherein the botanical material is a material from a plant in the tobacco family.

6. The aerosol generating composition according to claim 4 wherein the botanical material is a material from cannabis.

7. The aerosol generating composition according to claim 4 wherein the botanical material is rooibos.

8. The aerosol generating composition according to claim 4 further comprising one or more binders and optionally a filler.

9. The aerosol generating composition according to claim 8, wherein the one or more binders consists of one or more gelling agent(s).

10. The aerosolizable formulation according to claim 1 wherein the modulator comprises at least
(i) one or more of a TRPM8 agonist, a TRPV1 agonist, and a TRPV3 agonist; and
(ii) an TRPA1 antagonist or a TRPA1 inhibitor.

11. The aerosolizable formulation according to claim 1 wherein the modulator comprises at least
(i) a TRPM8 agonist or a TRPV1 agonist; and
(ii) a TRPA1 antagonist or a TRPA1 inhibitor.

12. The aerosolizable formulation according to claim 1 wherein the modulator comprises at least
(i) a TRPM8 agonist and a TRPV1 agonist; and
(ii) a TRPA1 antagonist or a TRPA1 inhibitor.

13. The aerosolizable formulation according to claim 1 wherein
(1) the TRPA1 antagonist binds with one or more sites selected from Arg852, Gln979, His983, Ile858, Leu982, Met978, Trp711, Val861, Val967, Ala836, Gln940, Ile837, Leu847, Leu848, Leu863, Leu867, Leu871, Met844, Phe841, Phe884, Phe947, Ser887, Tyr840; and combinations thereof, such as the TRPA1 antagonist binds with one or more sites selected from Arg852, Gln979, His983, Ile858, Leu982, Met978, Trp711, Val861, Val967; and combinations thereof; and/or
(2) the TRPA1 agonist binds with one or more sites selected from Ala836, Gln940, Ile837, Leu847, Leu848, Leu863, Leu867, Leu871, Met844, Phe841, Phe884, Phe947, Ser887, Tyr840; and combinations thereof; and/or
(3) the TRPM8 agonist binds with one or more sites selected from Arg1007, Arg841, Asn741, Asp781, Ile845, Leu1000, Phe738, Tyr1004, Tyr745, Val848; and combinations thereof; and/or
(4) the TRPV1 agonist binds with one or more sites selected from Ala548, Ala568, Ala667, Asn553, Glu572, Ile571, Ile663, Leu517, Leu555, Leu664, Leu671, Met549, Phe518, Phe545, Phe593, Ser514, Thr552, Tyr513, Tyr556; and combinations thereof; and/or
(5) the TRPV3 agonist binds with one or more sites selected from Arg693, His430, His426, His417, Leu420, Leu694, Leu429, Trp433, Thr421; and combinations thereof; and/or
(6) the TRPA1 inhibitor binds with one or more sites selected from Leu867, Leu870, Leu871, Ile946, Ser873 Thr873, Thr874, Phe944, Val948, Phe877, Ile878, Leu880, Leu881, Met912, Phe909, Thr908, Ile906, Ile905, Ile950, Leu956, Val942, Met953, Leu952, Phe884; and combinations thereof.

14. The aerosolizable formulation according to claim 1 wherein the active agent is at least nicotine and/or wherein the aerosolizable formulation composition further comprising at least one acid.

15. The aerosolizable formulation according to claim 1 wherein the modulator is at least caryophyllene oxide, benzyl cinnamate, or a mixture thereof.

16. The aerosolizable formulation according to claim 1 wherein the modulator is caryophyllene oxide and benzyl cinnamate.

17. The aerosolizable formulation according to claim 16 comprising caryophyllene oxide in an amount of 0.01 to 20 wt. % based on the weight of the aerosolizable formulation and benzyl cinnamate in an amount of 0.01 to 20 wt. % based on the weight of the aerosolizable formulation or aerosol generating composition.

18. A consumable for use in a non-combustible aerosol provision device, the consumable comprising an aerosolizable formulation as defined in claim 1.

19. A non-combustible aerosol provision system comprising the consumable of claim 18 and a non-combustible aerosol provision device, the non-combustible aerosol provision device comprising an aerosol-generation device arranged to generate aerosol from the consumable when the consumable is used with the non-combustible aerosol provision device.

20. (canceled)

21. A process for forming an aerosol comprising
(a) at least one active agent;
(b) an aerosol former material; and
(c) a modulator selected from a TRPA1 agonist, a TRPM8 agonist, a TRPV1 agonist, a TRPV3 agonist, a TRPA1 antagonist, a TRPA1 inhibitor and combinations thereof, the process comprising one of
(A) aerosolizing an aerosolizable formulation comprising
(a) at least one active agent;
(b) an aerosol former material; and
(c) a modulator selected from a TRPA1 agonist, a TRPM8 agonist, a TRPV1 agonist, a TRPV3 agonist, a TRPA1 antagonist, a TRPA1 inhibitor and combinations thereof
or
(B) aerosolizing an aerosolizable formulation comprising
(a) at least one active agent; and
(b) a carrier selected from water, propylene glycol, glycerol and mixtures thereof; and
(c) a modulator selected from a TRPA1 agonist, a TRPM8 agonist, a TRPV1 agonist, a TRPV3 agonist, a TRPA1 antagonist, a TRPA1 inhibitor and combinations thereof;
or
(C) aerosolizing an aerosolizable formulation comprising
(a) at least one active agent;
(b) aerosol former material;
(c) a modulator selected from a TRPA1 agonist, a TRPM8 agonist, a TRPV1 agonist, a TRPV3 agonist, a TRPA1 antagonist, a TRPA1 inhibitor and combinations thereof; and
(d) one or more binders;
or
(D) aerosolizing an aerosol generating composition comprising
(a) botanical material;
(b) an aerosol former; and
(c) a modulator selected from selected from a TRPA1 agonist, a TRPM8 agonist, a TRPV1 agonist, a TRPV3 agonist, a TRPA1 antagonist, a TRPA1 inhibitor and combinations thereof.

22. The process according claim 21 wherein the modulator is at least caryophyllene oxide, benzyl cinnamate, or a mixture thereof.

23. A kit comprising
(1) a first component comprising
(i) a first container; and
(ii) an aerosolizable formulation, comprising
(a) at least one active agent; and
(b) aerosol former material;
(2) a second component comprising
(i) a second container; and
(ii) a modulator formulation comprising a modulator selected from a TRPA1 agonist, a TRPM8 agonist, a TRPV1 agonist, a TRPV3 agonist, a TRPA1 antagonist, a TRPA1 inhibitor and combinations thereof.

24. The kit according claim 23 wherein the modulator is at least caryophyllene oxide, benzyl cinnamate, or a mixture thereof.

25. An electronic aerosol provision system comprising:
(i) an aerosol-generator for aerosolizing a formulation for inhalation by a user of the electronic aerosol provision system;
(ii) a power supply comprising a cell or battery for supplying power to the aerosol-generator
(iii) an aerosolizable formulation, comprising at least
(a) at least one active agent;
(b) aerosol former material; and
(c) a modulator selected from a TRPA1 agonist, a TRPM8 agonist, a TRPV1 agonist, a TRPV3 agonist, a TRPA1 antagonist, a TRPA1 inhibitor and combinations thereof.

26. The electronic aerosol provision system according claim 25 wherein the modulator is at least caryophyllene oxide, benzyl cinnamate, or a mixture thereof.

27-28. (canceled)

29. An aerosolizable formulation or aerosol formulation comprising
(a) at least one active agent;
(b) a carrier selected from water, propylene glycol, glycerol and mixtures thereof; and
(c) a modulator selected from a TRPA1 agonist, a TRPM8 agonist, a TRPV1 agonist, a TRPV3 agonist, a TRPA1 antagonist, a TRPA1 inhibitor and combinations thereof.

30. The aerosolizable formulation or aerosol formulation according to claim 29 wherein the modulator comprises at least one or more of a TRPA1 agonist, a TRPM8 agonist, a TRPV1 agonist, and a TRPV3 agonist.

31. The aerosolizable formulation or aerosol formulation according to claim 29 wherein the modulator comprises at least a TRPA1 antagonist or a TRPA1 inhibitor.

32. The aerosolizable or aerosol formulation according to claim 29 wherein the modulator comprises at least
(i) one or more of a TRPM8 agonist, a TRPV1 agonist, and a TRPV3 agonist; and
(ii) an TRPA1 antagonist or a TRPA1 inhibitor.

33. The aerosolizable or aerosol formulation according to claim 29 wherein the TRPA1 antagonist or a TRPA1 inhibitor has a stronger binding affinity to the TRPA1 receptor than the binding affinity of the one or more of a TRPM8 agonist, a TRPV1 agonist, and a TRPV3 agonist to their respective receptor.

34. The aerosolizable formulation or aerosol formulation according to claim 29 wherein the modulator comprises at least
(i) a TRPM8 agonist or a TRPV1 agonist; and
(ii) a TRPA1 antagonist or a TRPA1 inhibitor.

35. The aerosolizable formulation or aerosol formulation according to claim 29 wherein the modulator comprises at least
(i) a TRPM8 agonist and a TRPV1 agonist; and
(ii) a TRPA1 antagonist or a TRPA1 inhibitor.

36. The aerosolizable formulation or aerosol formulation according to claim 29 wherein
(1) the TRPA1 antagonist binds with one or more sites selected from Arg852, Gln979, His983, Ile858, Leu982, Met978, Trp711, Val861, Val967, Ala836, Gln940, Ile837, Leu847, Leu848, Leu863, Leu867, Leu871, Met844, Phe841, Phe884, Phe947, Ser887, Tyr840; and combinations thereof, such as the TRPA1 antagonist binds with one or more sites selected from Arg852, Gln979, His983, Ile858, Leu982, Met978, Trp711, Val861, Val967; and combinations thereof; and/or
(2) the TRPA1 agonist binds with one or more sites selected from Ala836, Gln940, Ile837, Leu847, Leu848, Leu863, Leu867, Leu871, Met844, Phe841, Phe884, Phe947, Ser887, Tyr840; and combinations thereof; and/or
(3) the TRPM8 agonist binds with one or more sites selected from Arg1007, Arg841, Asn741, Asp781, Ile845, Leu1000, Phe738, Tyr1004, Tyr745, Val848; and combinations thereof; and/or
(4) the TRPV1 agonist binds with one or more sites selected from Ala548, Ala568, Ala667, Asn553, Glu572, Ile571, Ile663, Leu517, Leu555, Leu664, Leu671, Met549, Phe518, Phe545, Phe593, Ser514, Thr552, Tyr513, Tyr556; and combinations thereof; and/or
(5) the TRPV3 agonist binds with one or more sites selected from Arg693, His430, His426, His417, Leu420, Leu694, Leu429, Trp433, Thr421; and combinations thereof;
and/or
(6) the TRPA1 inhibitor binds with one or more sites selected from Leu867, Leu870, Leu871, Ile946, Ser873 Thr873, Thr874, Phe944, Val948, Phe877, Ile878, Leu880, Leu881, Met912, Phe909, Thr908, Ile906, Ile905, Ile950, Leu956, Val942, Met953, Leu952, Phe884; and combinations thereof.

37. The aerosolizable formulation or aerosol formulation according to claim 29 wherein the modulator is at least caryophyllene oxide, benzyl cinnamate, or a mixture thereof.

38. The aerosolizable formulation or aerosol formulation according to claim 29 wherein the modulator is a mixture of caryophyllene oxide and benzyl cinnamate.

39. The aerosolizable formulation or aerosol formulation according to claim 29 wherein the active agent is at least nicotine.

40. The aerosolizable formulation or aerosol formulation according to claim 29 wherein the active agent is at least a cannabinoid.

41. The aerosolizable formulation or aerosol formulation according to claim 29 wherein the active agent is present in an amount of no greater than 6 wt. % based on the aerosolizable formulation or aerosol formulation.

42. The aerosolizable formulation or aerosol formulation according to claim 41 wherein the active agent is present in an amount of from 0.01 to 5 wt. % based on the aerosolizable formulation or aerosol formulation.

43. The aerosolizable formulation or aerosol formulation according to claim 29 further comprising at least one acid.

44. The aerosolizable formulation or aerosol formulation according to claim 43 wherein the acid is selected from the group consisting of acetic acid, lactic acid, formic acid, citric acid, benzoic acid, pyruvic acid, levulinic acid, succinic acid, tartaric acid, sorbic acid, propionic acid, phenylacetic acid, salicylic acid, malic acid, caffeic acid, fumaric acid, nicotinic acid, o-toluic acid, m-toluic acid, p-toluic acid, capric acid, glucono delta-lactone, gluconic acid, malonic acid, phosphoric acid, 4-hydroxyphenylacetic acid and mixtures thereof.

45. The aerosolizable formulation or aerosol formulation according to claim 43 wherein the acid is selected from the group consisting of salicylic acid, malic acid, citric acid, succinic acid, caffeic acid, fumaric acid, nicotinic acid, o-toluic acid, m-toluic acid, p-toluic acid, capric acid, glucono delta-lactone, pyruvic acid, sorbic acid, levulinic acid, tartaric acid, gluconic acid, malonic acid, phosphoric acid, 4-hydroxyphenylacetic acid and mixtures thereof.

46. The aerosolizable formulation or aerosol formulation according to claim 43 wherein the total content of acid present in the formulation is no greater than 1.0 mole equivalents based on the nicotine.

47. The aerosolizable formulation or aerosol formulation according to claim 43 wherein the total content of acid present in the solution is no less than 0.1 mole equivalents based on the nicotine.
